(19) United States
(12) Patent Application Publication
(10) Pub. No.: US 2026/0002195 A1
(43) Pub. Date: Jan. 1, 2026

(54) METHOD FOR ANALYZING BLOOD STORED FOR LATER ANALYSIS OF CELL FREE DNA

(71) Applicants: Stephen Little, Thresfield (GB); Michael D. Risley, Cheshire (GB); Joanne N. Mason, Cambridge (GB); Francois Rousseau, Québec (CA)

(72) Inventors: Stephen Little, Thresfield (GB); Michael D. Risley, Cheshire (GB); Joanne N. Mason, Cambridge (GB); Francois Rousseau, Québec (CA)

(21) Appl. No.: 18/729,856

(22) PCT Filed: Jan. 17, 2023
(86) PCT No.: PCT/IB2023/000022

§ 371 Date: Sep. 11, 2025

Related U.S. Application Data
(60) Provisional application No. 63/300,256, filed on Jan. 17, 2022.

(51) Int. Cl. C12Q 1/6806; C12Q 1/6886

(52) U.S. Cl. CPC C12Q 1/6806; C12Q 1/6886

(57) ABSTRACT

The present invention relates to storing and testing for abnormalities in cell free DNA (cfDNA) from blood, such as in maternal blood or blood for cancer screening.

### 1. FIELD OF THE INVENTION

The present invention relates to storing and testing for abnormalities in cell free DNA (cfDNA) from plasma, such as plasma in maternal blood or in blood in a cancer patient or in a patient being screened for cancer.

### 2. BACKGROUND

Since its introduction into clinical care in 2011 (See Ehrich, 2011 and Palomaki, 2011), noninvasive prenatal test (NIPT) has provided many pregnant women in different countries with information about their risk for fetal aneuploidies. Whatever the platform used or the protocol followed, most laboratories will report between 0 and 25% failure rate. (Badeau, 2017.) Failures are usually due to a fetal fraction too low to be reliable. (Blais, 2018.) Some reasons for a low fetal fraction are known, such as early gestational age, a high BMI (body mass index), presence of some fetal or placental anomaly, a T18 and maternal age. (Hou, 2019; Ashoor, 2013.)

Increasing gestational age is associated with an increase of fetal fraction, however screening tests are useful at the beginning of a pregnancy and the increased fetal fraction is mostly observed late in pregnancy. (Wang, 2013.) At the molecular level, fetal cfDNA can be distinguished from maternal cfDNA by the fact that fragments are shorter (Fan, 2010; Shi, 2020), hypermethylated and enriched at specific genomic locations (Wang, 2013). Recently, several studies reported that the use of high concentration agarose gel electrophoresis to select the shortest fragments of a library prepared from cfDNA before sequencing increased the fetal fraction meaningfully (Welker, 2021; Qiao, 2019; Xue, 2020; Qiao, 2019; Hu, 2019). The same procedure appeared also to improve the detection of rare mutated alleles in the context of cancer. (Underhill, 2021.)

Tumor circulating cell-free DNA was also shown to be shorter than cfDNA from healthy cells. (Jiang, 2015.) For both applications, different systems were used to isolate or enrich these short fragments with electrophoresis using precast gels such as 2% E-gel EX from Invitrogen (Thermofisher), 2-3% agarose cassettes from BluePippin (Sage Bioscience) and 3% agarose cassettes from Yourgene Health Canada, Inc. (Underhill, 2021.)

To obtain an adequate proportion of cfDNA for meaningful sequencing, plasma must be prepared with some caution to avoid contamination with host genomic DNA. Blood samples need to be processed within 4-8 hours after collection and an additional high-speed centrifugation step for plasma preparation is preferred by some. (Barrett, 2011.) However, this is not always possible because collection sites are not always equipped to carry out high-speed centrifugation of plasma samples prior to freezing the samples for shipping to a central testing laboratory. The solution adopted by most phlebotomy sites is to use specialized tubes designed to maintain the integrity of cfDNA such as by using cross linking agents to stabilize the cell membrane. An example of such specialized tubes is Streck® blood collection tubes. Not using a specialized tube and storing blood for an extended period causes an increase in cfDNA over time due to the breakdown of genomic material. (Fernando 2018.) Specialized tubes stabilize the blood sample, allowing time for the sample to be transferred to a laboratory with the equipment to process the sample. However, these specialized tubes are expensive.

The use of vacutainer K₂EDTA with gel to collect plasma for molecular diagnostics was recently reported that could yield NIPT-grade cfDNA (Giroux, 2021.) The use of these tubes followed by filtration was shown to be cost effective and efficient to reduce time consuming steps in the laboratory. However, despite the fact that most sites can centrifuge a blood tube, many sites are not equipped with facility and staff to open the blood tube and filter the plasma.

Therefore, a need exists for alternatives for storing whole blood samples and processing blood for meaningful sequence analysis of cfDNA.

### 3. SUMMARY OF THE INVENTION

Embodiments of the present disclosure relate to a method for analyzing nucleic acid sequences obtained from a blood sample to provide sequence information that comprises: collecting a blood sample into a blood collection device, wherein said blood collection device does not comprise a fixative; storing the blood without fixative prior to isolating the cell-free DNA from the plasma at a temperature greater than −20° C. and less than 35° C. (such as room temperature or refrigeration temperatures); separating plasma from blood cells present in the blood sample either before or after storage; isolating cell-free DNA from the plasma greater than 24 hours after blood collection from a subject; separating the cell free DNA by size of the cell free DNA and isolating the cell free DNA that is less than 300 bp; and analyzing nucleic acid sequences of the isolated cell free DNA to detect sequence information. The blood can be stored for an extended period without a fixative before the cfDNA is isolated from the plasma. This time period can be up to 3, 4, 5, 6, 7, 8, or 9 days after blood collection.

A suitable collection device for use with the invention can comprise a container having only one or more compositions therein wherein the one or more compositions consists of one or more anti-coagulants and a metal ion selected from potassium, lithium, and sodium. An example of such devices is a K₂EDTA tube. Alternatively, the blood collection device can comprise a container having only one or more compositions therein wherein the one or more compositions consists of one or more anti-coagulants and a metal ion selected from potassium, lithium, and sodium; and a separator gel. In other embodiments, the collection device can comprise a container having only one or more compositions therein wherein the one or more compositions comprises or consists of a coagulant.

As mentioned above the separation step can be used to isolate cfDNA that is less than 300 bp in the sample of isolated cfDNA. In some embodiments, cfDNA that is isolated in the size separation step is at or less than 185 bp or at or less than 165 bp. In other embodiments, cfDNA that is isolated in the size separation step is at or less than 155 bp, or at or less than 150 bp. In further embodiments, the portion of the cfDNA isolated from the plasma sample is at or greater than 50 bp or at or greater than 80 bp. The separation step can be performed by any number of suitable polynucleotide size selection techniques known in the field, including gel electrophoresis, chromatography, bead-based separation, or a membrane filter with a pore size that impedes passage of polynucleotides of size larger than the desired threshold.

After size selection, the nucleic acid sequences of the isolated cell free DNA can be sequenced and analyzed to determine the presence of a genetic anomaly in fetal DNA or to determine sequence information about or confirm the presence of cancerous cells in a subject based on the presence of one more genetic markers associated with tumorgenicity or malignancy. In order to obtain sequence information about the isolated cfDNA, a sequence library may need to be prepared, and the cfDNA may need to be amplified. Once size selection and library preparation is complete, the sample can be analyzed to obtain sequence information. Sequence information can be analyzed to detect mutations and chromosomal abnormalities including but not limited to translocation, transversion, monosomy, trisomy, and other aneuploidies, deletion, insertion, methylation, amplification, fragment, translocation, and rearrangement or chromathripsis. Sequence information can also be used to detect any alteration in gene sequence as compared to a reference sequence.

Another aspect of this disclosure is a sequence validity test that can detect for abnormal performance of the size selection step or unacceptable sample degradation. In a method involving size selection of a DNA sample comprising cfDNA and the sequencing of the sample DNA after size selection; the method can comprise a sequence validity test of sequence information obtained from the sequencing step. The validity test comprises creating a first normalised fragment size profile from the sequencing information associated with the sample, comparing one or more values within the fragment size profile against one or more of corresponding value ranges obtained from a reference parameter set comprising a plurality of valid fragment size profiles; and accepting the sequence information and the analysis results thereof if one of the one or more values within the fragment size profile associated with the sample is within the corresponding value range or rejecting the sequence information if one of the one or more values within the fragment size profile associated sample is outside of the corresponding value range. The validity test may also comprise calculating a first relative fragment size frequency value from the sequencing information associated with the sample; comparing the first relative fragment size frequency value to a reference relative fragment size frequency value set comprising a plurality of valid relative fragment size frequencies, and accepting the sequence information and the analysis results thereof if the first fragment size frequency associated with the sample is within a specified range of values within the reference set or rejecting the sequence information and the analysis results thereof if the first fragment size frequency associated with the sample is outside a specified range of values within the reference set.

### 4. BRIEF DESCRIPTION OF THE FIGURES

FIG. 1 depicts a plot showing the fetal fraction of various samples at various time points for samples stored in a plasma preparation tube or a Streck® tube and stored for 2-9 days.

FIG. 2A depicts a plot showing the fetal fraction calculated from the reduction of chromosome X fragments in male pregnancy. FIG. 2B depicts Fetal fraction evaluated with the number of chromosome Y fragments for males only.

FIG. 3 depicts a plot illustrating the size of the sequenced fragments calculated from the mapping of paired-ends sequencing. In grey fragments before the size-selection and in blue after size-selection

FIG. 4 depicts a study schematic of the study described in Example 3. Upon enrollment each participant donated 20 mL of blood. Blood was collected in 5 mL EDTA vacutainers, and transported to storage and processing to a destination laboratory. Blood was stored within their vacutainers at ambient temperature for 0-8 hours, 72 hours, 120 hours, or 168 hours before processing by double centrifugation to obtain EDTA plasma. Plasma was stored at −80° C. and processed at intervals once enough samples had been collected for a run.

FIG. 5 depicts one-way ANOVA of the raw fetal fraction estimate for the 268 valid samples after sequencing, showing the fetal fraction estimate data by Time Point, where the mean for all timepoints is shown by the horizontal line, green represents ANOVA and blue represents ±standard deviation, and the ANOVA, mean and standard deviation analysis. Time Point 1=0-8 hours, Time Point 2=72 hours, Time Point 3=120 hours and Time Point 4=168 hours

FIG. 6. Means, standard deviations and Welch ANOVA of the 275 valid libraries for this study, plus the sample M056 (Time Point 1; 2.49 ng/μL) from Run 5. Post-PCR library quantification (ng/μL) data is presented by Time Point. Note: the four equal variances tests demonstrate that the variances are not equal for data across the Time Points; hence a Welch ANOVA was performed in place of ANOVA. Time Point 1=0-8 hours, Time Point 2=72 hours, Time Point 3=120 hours and Time Point 4=168 hours. Welch ANOVA P<0.0001 shows significant variance in library concentration data across the four Time Points in the study (P<0.05)

FIG. 7A: Size profile distribution and frequency for each Time Point for patient sample M047 where: Time Point 1=Red/TP1 (YGL013-6), Time point 2=Orange/TP2 (YGL015-6), Time Point 3=Blue/TP3 (YGL038-6) and Time Point 4=Green/TP4 (YGL012-6)

FIG. 7B. Size profile distribution and frequency for each Time Point for patient sample M100 where: Time Point 1=Red/TP1 (YGL026R-12), Time point 2=Orange/TP2 (YGL003-12), Time Point 3=Blue/TP3 (YGL021-12) and Time Point 4=Green/TP4 (YGL012-12).

### 5. DETAILED DESCRIPTION

A method of storing blood containing cfDNA to be sequenced is described herein. In accordance with the present disclosure, whole blood samples are stored in a blood collection device lacking a fixative for an extended period of time, greater than 24 hours, prior to the cfDNA being isolated from the blood or plasma for obtaining sequence information. The method comprises performing a size selection step of the isolated DNA to select for cfDNA that is less than a certain size, for example, less than or equal to 185 bp, prior to obtaining sequence information on the size selected cfDNA. By selecting for cfDNA having a nucleotide length of less than or equal to, e.g., 185 bp, the fraction of fetal cfDNA that is in the cfDNA sample is increased, thereby increasing the quality of the sample for meaningful sequence analysis.

### 5.1. Definitions

“Blood sample” herein refers to a whole blood sample that has not been fractionated or separated into its component parts.

“Fixing” is a technique that helps to maintain the structure of cells and/or sub-cellular components such as cell organelles (e.g., nucleus) in a blood sample. Fixing modifies the chemical or biological structure of cellular components by, e.g., cross-linking them. Fixing impedes lysis of whole cells and cellular organelles, thereby inhibiting release of cellular nucleic acids into a surrounding medium. For example, fixing may inhibit nuclear DNA from white blood cells releasing into a plasma fraction during centrifugation of whole blood.

“Fixative” is an agent used for fixing a blood sample.

“Sequence of interest” refers to a nucleic acid sequence that is associated with a difference in sequence representation in healthy versus diseased individuals. A sequence of interest can be a sequence on a chromosome that is misrepresented, i.e., over- or under-represented, in a disease or genetic condition. A sequence of interest may be a portion of a chromosome, i.e., chromosome segment, or a whole chromosome. For example, a sequence of interest can be a chromosome that is over-represented in an aneuploidy condition, or a gene encoding a tumor-suppressor that is under-represented in a cancer. Sequences of interest include sequences that are over- or under-represented in the total population, or a subpopulation of cells of a subject. May also be referred to as a “marker” for a genetic genotype associated with a particular phenotype, such as disease.

“Sequence tag” refers to a sequence of at least 30 bp read from a strand of cfDNA. In some embodiments, the tag has been specifically assigned, i.e., mapped, to a larger sequence, e.g., a reference genome, by alignment.

### 5.2. Method for Increasing the Fraction of a Portion of cfDNA and Analyzing the Same

Embodiments of the present disclosure are directed to a method of increasing the fraction of a portion of cfDNA from plasma, such as from a maternal blood sample or a blood sample for a cancer diagnostic, and analyzing the nucleic acid sequences of the fraction to provide sequence information. The method involves collecting a blood sample into a blood collection device. The blood collection device does not comprise a fixative. In some embodiments, the blood collection device consists of a container with one or more anti-coagulants and a metal ion selected from potassium, sodium, and lithium. In some embodiments, the blood collection device consists of a container with one or more anti-coagulants and a metal ion, such as potassium, sodium, or lithium, and a separator gel.

The blood sample is centrifuged before or after storage without a fixative to separate the plasma from the blood cells. Before or after the plasma is separated, the blood sample can be stored at a temperature in a range −20° C. to 35° C., such as at room or ambient temperature, at refrigeration temperatures, or between room temperature and refrigeration temperatures until the sample can be further processed. When the sample is able to be processed between 24-216 hours, e.g., 48/72 to 216 hours or 48/72 to 192 hours or 48/72 to 168 hours or 48/72 to 144 hours after blood collection, the cfDNA is isolated from the plasma. In order to increase the fraction of a portion of the cfDNA, the cfDNA is separated by size, such as by gel electrophoresis and the cfDNA that less than 300 bp (e.g., less than 185, 165, 160, 155, or 150 bp) is isolated. Either before or after size selection, the cfDNA sample can be prepared for sequencing by optionally amplifying and generating a sequence library comprising the cfDNA. The size selected and isolated cfDNA can then be analyzed to detect sequence information. The steps described herein may be performed by one entity or multiple entities.

### 5.2.1 Blood Collection Device

A blood sample is collected in a collection device. The collection device may be an evacuated collection container, usually a tube. In accordance with the present disclosure, the blood collection device does not contain a fixative. Examples of suitable blood collection devices include K2 or K3 EDTA blood collection tubes with or without a separator gel.

As defined above, a fixative is an agent used for fixing a blood sample. Examples of fixatives include aldehydes (e.g., formaldehyde, glutaraldehyde, and derivatives thereof), formalin alcohols, sulfhydryl reactive cross-linkers, carbohydrate reactive cross-linkers, carboxyl reactive cross-linkers, photoreactive cross-linkers, cleavable cross-linkers, AEDP (3-[(2-aminoethyl)dithio]propionic acid⋅HCl), APG (p-Azidophenyl Glyoxal monohydrate), BASED, BM(PEO)₃ (1,8-bis-MaleiMido-PEO₃), BM(PEO)₄ (1,8-bis-MaleiMido-PEO₄), BMB (1,4-bismaleimidobutane), BMDB (1,4-Bis-Maleimidyl-2,3-dihydroxybutane), BMH (Bis-Maleimidohexane), BMOE (Bis-Maleimidoethane), BS3 (Bis(sulfosuccinimidyl)suberate), BSOCOES (Bis(2-[succinimidoxycarbonyloxy]ethyl)sulfone), DFDNB (1-5-Difluoro-2,4-dinitrobenzene), DMA (Dimethyl adipimidate⋅2HCl), DMP (Dimethyl pimelimidate⋅2HCl), DMS (Dimethyl suberimidate⋅2HCl), DPDPB (1,4-Di-(3′-[2′pyridyldithio]propionamido) butane), DSG (Disuccinimidyl glutarate), DSP (Dithiobis(succimidylpropionate) (Lomant's Reagent)), DSS (Disuccinimidyl suberate), DST (Disuccinimidyl tartarate), DTBP (Dimethyl 3,3′-dithiobispropionimidate⋅2HC), DTME (Dithiobis-maleimidoethane), DTSSP (3,3′-Dithiobis(sulfosuccinimidylpropionate)), EGS (Ethylene glycol bis(succinimidylsuccinate)), HBVS (1,6-Hexane-bis-vinylsulfone), sulfo-BSOCOES, Sulfo-DST, and Sulfo-EGS.

Other fixatives can be a stabilizer of cell membrane such as DMAE (dimethylaminoethanol), cholesterol, cholesterol derivatives, high concentrations of magnesium, vitamin E, and vitamin E derivatives, calcium, calcium gluconate, taurine, niacin, hydroxylamine derivatives, bimoclomol, sucrose, astaxanthin, glucose, amitriptyline, isomer A hopane tetral phenylacetate, isomer B hopane tetral phenylacetate, citicoline, inositol, vitamin B, vitamin B complex, cholesterol hemisuccinate, sorbitol, calcium, coenzyme Q, ubiquinone, vitamin K, vitamin K complex, menaquinone, zonegran, zinc, ginkgo biloba extract, diphenylhydantoin, perftoran, polyvinylpyrrolidone, phosphatidylserine, tegretol, PABA, disodium cromglycate, nedocromil sodium, phenyloin, zinc citrate, mexitil, dilantin, sodium hyaluronate, or polaxamer 188.

Other fixatives are formaldehyde releaser preservative agent such as one selected from the group consisting of: diazolidinyl urea, imidazolidinyl urea, dimethoylol-5,5-dimethylhydantoin, dimethylol urea, 2-bromo-2.-nitropropane-1,3-diol, oxazolidines, sodium hydroxymethyl glycinate, 5-hydroxymethoxymethyl-1-1aza-3,7-dioxabicyclo [3.3.0]octane, 5-hydroxymethyl-1-1aza-3,7dioxabicyclo[3.3.0]octane, 5-hydroxypoly[methyleneoxy]methyl-1-1aza-3,7dioxabicyclo[3.3.0]octane, quaternary adamantine and any combination thereof.

In a further embodiment, other than EDTA and/or heparin, the blood collection device may not contain agents that reduce DNA degradation, such as a nuclease inhibitor. Examples of nuclease inhibitors include diethyl pyrocarbonate, ethanol, aurintricarboxylic acid (ATA), formamide, vanadyl-ribonucleoside complexes, macaloid, proteinase K, hydroxylamine-oxygen-cupric ion, bentonite, ammonium sulfate, dithiothreitol (DTT), beta-mercaptoethanol, cysteine, dithioerythritol, tris(2-carboxyethyl) phosphene hydrochloride, and a divalent cation such as Mg+2, Mn+2, Zn+2, Fe+2, Ca+2, Cu+2. Other agents that reduce DNA degradation may also be excluded from the device, such as zinc chloride, guanidine-HCl, guanidine isothiocyanate, N-lauroylsarcosine, and Na-dodecylsulphate.

The present disclosure contemplates excluding any specific combination of the above fixatives or any specific combination of the above fixatives and/or agents that reduce DNA degradation, such as nuclease inhibitors.

In some embodiments, the collection device contains an anti-coagulant. An anti-coagulant can be selected from ethylenediaminetetraacetic acid (EDTA), heparin, citrate, oxalate, and any combination thereof. In some embodiments, the anticoagulant can be spray dried on the wall of the collection device. In other embodiments, the anticoagulant is a liquid solution in the device. Suitable solvents include water, saline, dimethylsulfoxide, alcohol, and mixtures thereof. In embodiments, the collection device comprises a container having only one or more compositions, where the one or more compositions consist of an anti-coagulant and a metal ion, such as those selected from alkali or alkaline earth metals. In particular, the metal ion can be selected from calcium, sodium, lithium, and potassium.

In embodiments with EDTA, the EDTA can be chelated with a metal ion selected from potassium, lithium, and sodium, and is typically potassium. In some embodiments, the blood collection device comprises a container having only one or more compositions, where the one or more compositions consist of chelated EDTA.

In some embodiments, the collection device contains a separator gel. Such gels are commonly used in serum separation tubes (SSTs) for plasma preparation (see e.g., BD Vacutainer® SST™ and PST™, Becton, Dickinson, and Company). The gel will have a density intermediate to that of the cells in the blood and the liquid phase of the blood (densities of 1.09 and 1.03 g/cm3 respectively). By virtue of its density, the separator gel physically separates the liquid component (plasma) of the blood from the erythrocytes and white blood cells after centrifugation and acts as barrier between the liquid phase and the cells. The separator gel is an inert gel, such as a polyester gel or silicone gel. Other suitable gels include sorbitol-based gelator in a diacrylate oligomer.

In some embodiments, the blood collection device comprises a container having only one or more compositions, where the one or more compositions consist of an anti-coagulant and a metal ion, such as chelated EDTA, and a separator gel.

In some embodiments, the collection device contains a coagulant.

### 5.2.2 Storage of Blood Collection Device and Removal of Large Molecules From Plasma

In some embodiments, after the blood sample is collected from a patient, the blood can be stored at temperatures between −20° C.-35° C., such as standard ambient temperature (25° C.), room temperature (20-24° C.), refrigeration temperatures (1-8° C.), or any temperature in between for a period of up to 7-9 days prior to centrifugation to separate the plasma from the cell fraction and extraction of DNA. The storage time prior to centrifugation can be 2, 3, 4, 5, 6, 7, 8, or 9 days. The storage temperature can be 1° C., 2° C., 3° C., 4° C., 5° C., 6° C., 7° C., 8° C., or some value in between or range thereof. In other embodiments, the storage temperature can be within the range of 9-23° C., 20-25° C., 25-30° C. or 30-35° C. In some embodiments, the storage time does not exceed 7 days or is between 2-5 days, 2-6 days, 2-7 days, 3-5 days, 3-6 days, 4-6 days, 3-7 days, or 4-7 days. In some embodiments, the storage time is between 2-7 days or 3-7 days and is stored at refrigeration temperature, room temperature, or some temperature therebetween.

Once removed from storage, such as refrigerated storage, the sample can be fractionated to remove the larger size particulates. Techniques for such are described in Pös et al., Int. J. Mol. Sci. 2020, 21, 8634, entitled “Technical and Methodological Aspects of Cell-Free Nucleic Acids Analyzes,” which is hereby incorporated by reference. In one embodiment, this is done by centrifugation. The centrifugation can be a one-step or a two-step process. In some embodiments, the centrifugation is conducted at refrigeration temperatures. In other embodiments, it is at room temperature. For the first centrifugation step, the sample is subject to low speed centrifugation. The plasma is harvested from the low speed fractionated sample. For the second step, the plasma is subject to a low speed or high speed centrifugation step. The standard practice is a high speed step. The low speed centrifugation parameters are those commonly used for separating plasma from the cellular components in a blood sample. For example, for the low speed step, the spin speed can be between 1,000 to 2,000×g, and the spin time can be 8, 9, 10, 11, 12 minutes or some time in between or range thereof. In some embodiments, the spin speed can be 1,500-1,900×g or 1,600-1,800×g and the spin time is 9-11 minutes or 10 minutes. For the high speed step, the spin speed can be approximately 3-10 times that of the low speed step for the same amount of time. For example, the high speed centrifugation can be 3,000 to 18,000×g.

In lieu of a second centrifuging step, a microfiltration step can also be used to remove larger particles from the plasma. In some embodiments, the microfiltration step comprises a membrane separation process to remove the particles having an average molecular weight>200 kDa, >100 kDa, >75 kDa, or >65 kDa using membranes with a pore size less than 3, 2, or 1 μm. In some embodiments, the microfiltration membrane filter has a pore size of at or less than 0.55 μM, 0.50 μM, or 0.45 μM.

In other embodiments, the blood sample is collected in a collection device, such as with a collection device with a separator gel, and centrifuged shortly after collection at a low speed to separate the plasma from the cell fraction of the blood sample. In embodiments, this step can be done within 6-8 hours of sample collection and the collection device with the fractionated sample therein (i.e., immediate isolation of the plasma is not required) can be stored thereafter. Alternatively, the plasma can be removed from the collection device and stored in a second collection device that does not comprise a fixative and stored. The low speed centrifugation parameters are the same as those described above. The storage temperature and time of storage is the same as described above. Once removed from storage, the plasma can be removed from the collection device, if not removed prior to storage, and subject to a second centrifugation step or a filtration step in accordance with the parameters described above.

5.2.3 cfDNA Isolation

After a plasma fraction is collected as described above, the cfDNA is isolated/extracted from the plasma. Extraction is actually a multistep process that involves separating DNA from the plasma in a column or other solid phase binding matrix. The extracted cfDNA usually includes both maternal and fetal cfDNA. In some embodiments, the isolation can be performed by the following operations.
A. Denature and/or degrade proteins in plasma (e.g. contact with proteases) and add guanidine hydrochloride or other chaotropic reagent to the solution (to facilitate driving cfDNA out of solution)
B. Contact treated plasma with a support matrix such as a membrane or beads in a column or superparamagnetic particles with a coating to selectively bind nucleic acids. Such coatings can comprise silica, carboxyls, amines, imidazoles, or combinations thereof.
C. Wash the support matrix.
D. Release cfDNA from matrix with a release solution to recover the cfDNA for downstream process (e.g., indexed library preparation) and statistical analyses.

The first part of this cfDNA isolation procedure involves denaturing or degrading the nucleosome proteins and otherwise taking steps to free the DNA from the nucleosome. A typical reagent mixture used to accomplish this isolation includes a detergent, protease, and a chaotropic agent such as guanine hydrochloride. The protease serves to degrade the nucleosome proteins, as well as background proteins in the plasma such as albumin and immunoglobulins. The chaotropic agent disrupts the structure of macromolecules by interfering with intramolecular interactions mediated by non-covalent forces such as hydrogen bonds. The chaotropic agent also renders components of the plasma such as proteins negative in charge. The negative charge makes the medium somewhat energetically incompatible with the negatively charged DNA. The use of a chaotropic agent to facilitate DNA purification is described in Boom et al., “Rapid and Simple Method for Purification of Nucleic Acids”, J. Clin. Microbiology, v. 28, No. 3, 1990

After this protein degradation treatment, which frees, at least partially, the DNA coils from the nucleosome proteins, the resulting solution is passed through a column or otherwise exposed to a support matrix that selectively adheres the cfDNA in the treated plasma. The negative charge imparted to medium components facilitates adsorption of DNA in the pores of a support matrix.

After passing the treated plasma through the support matrix, the support matrix with bound cfDNA is washed to remove proteins and other unwanted components of the sample. After washing, the cfDNA is freed from the matrix and recovered. By way of example, in the case of amine- or imidazole-functionalized beads, in an acidic solution, cfDNA adsorbs to positively charged amine or imidazole moiety. In a basic solution, the amine and imidazole moieties are negatively charged and cfDNA is not adsorbed. Accordingly, to remove the unwanted components of the sample, the magnetic beads are immobilized with a magnet and the beads are washed with an acidic wash. After washing, the beads are separated from the magnet, and the cfDNA can be recovered by washing with a solution of higher pH. A similar process is used for carboxyl-coated beads. In the case of carboxyl-coated beads, cfDNA reversibly binds to the beads in the presence of polyethylene glycol (PEG) depending on the salt concentration therein.

These DNA extraction processes typically lose a significant fraction of the available DNA from the plasma. Support matrixes have a high affinity for cfDNA, which limits the amount of cfDNA that can be easily separated from the matrix. As a consequence, the yield of cfDNA extraction step can be quite low. Typically, the efficiency is well below 50% (e.g., it has been found that the typical yield of cfDNA is 4-12 ng/ml of plasma from the available ˜30 ng/ml plasma).

Commercially available kits for manual and automated separation of cfDNA can be used, such as QIAamp DNA Micro kit (Qiagen of Valencia, CA) or the NucleoSpin Plasma kit (Macherey-Nagel or Duren, DE). A process of isolation of cfDNA is described in U.S. Pat. No. 10,837,055, which is hereby incorporated by reference in its entirety.

### 5.2.4 Sequence Library Prep

The purified cfDNA obtained from the above isolation/extraction procedure can be used to prepare a library for sequencing. To sequence a population of double-stranded DNA fragments using massively parallel sequencing systems, the DNA fragments must be flanked by known adapter sequences. A collection of such DNA fragments with adapters at either end is called a sequencing library. Two examples of suitable methods for generating sequencing libraries from purified DNA are (1) ligation-based attachment of known adapters to either end of fragmented DNA, and (2) transposase-mediated insertion of adapter sequences.

Methods for generating a sequence library are described in US Publication No. US2007/0128624 entitled “Methods for preparing libraries of template polynucleotides” to Illumina Cambridge, Ltd., US Publication No. US2013/0123120 entitled “Highly Multiplex PCR Methods and Compositions” to Natera, Inc., J. F. Hess, et al., Library preparation for next generation sequencing: A review of automation strategies, Biotechnology Advances, Volume 41, 2020, 107537, ISSN 0734-9750, https://doi.org/10.1016/j.biotechadv.2020.107537, and Head, Steven R et al. Library construction for next-generation sequencing: overviews and challenges, BioTechniques vol. 56, 261-4, 66, 68, passim. 1 Feb. 2014, doi: 10.2144/000114133, all of which are hereby incorporated by reference in their entirety. A person of skill would appreciate that there are many suitable massively parallel sequencing techniques.

Sequencing libraries can be normalized to facilitate volumetric sample pooling using techniques known to a person of skill in the arts.

5.2.5 cfDNA Amplification

In some embodiments, the cfDNA present in the sample can amplified, either before or after preparing the sequence library. Amplification can be performed with any suitable technique such as methods based on polymerase chain reaction (PCR) or recombinase polymerase amplification (RPA). Such techniques are known to those in field. RPA is described in Lobato, et al., Recombinase polymerase amplification: Basics, applications and recent advances, Trends in analytical chemistry: TRAC vol. 98 (2018): 19-35. doi:10.1016/j.trac.2017.10.015, which is hereby incorporated by reference in its entirety.

To amplify a segment of DNA using PCR, the sample is first heated so the DNA denatures, or separates into two pieces of single-stranded DNA. Next, an enzyme called “Taq polymerase” synthesizes-builds-two new strands of DNA, using the original strands as templates. This process results in the duplication of the original DNA, with each of the new molecules containing one old and one new strand of DNA. Then each of these strands can be used to create two new copies, and so on, and so on. The cycle of denaturing and synthesizing new DNA is repeated. In some embodiments, 5, 6, 7, 8, 9, 10, 11, 12, 13, 14, 15, 16, 17, 18, 19, or 20 amplification cycles are performed. PCR is described in more detail in Lorenz, Todd C., Polymerase chain reaction: basic protocol plus troubleshooting and optimization strategies, Journal of Visualized Experiments, 63 e3998. 22 May. 2012, doi:10.3791/3998, which is hereby incorporated by reference in its entirety.

### 5.2.6 Size Selection

Before or after amplification, size selection is performed to increase the percentage of the fetal fraction in the sample thereby increasing its signal when being sequenced. The desired sequence length maximum of cfDNA to isolate by size selection can be 185 bp, 180 bp, 170 bp, 175 bp, 170 bp, 165 bp, 160 bp, 155 bp, 150 bp or 145 bp, or some value there between. In some embodiments, the desired sizes of cfDNA is a range 80-160 bp, 80-155 bp, 80-150 bp, 90-160 bp, 90-155 bp, 90-150 bp, 100-150 bp, 100-155 bp, 100-160 bp, 110-150 bp, 110-155 bp, 110-160 bp, 80-170 bp, or 80-185 bp. In some embodiments, the desired sizes of cfDNA is a range 100-200 bp, 150-250 bp, 200-300 bp, and 250-300 bp.

Size selection can be performed by any technique for separating macromolecules by size, such as gel electrophoresis, chromatography, bead binding matrix, or membrane filtration. In the case of gel electrophoresis, the desired size range can be recovered manually or by automation using an instrument, such as LightBench or NIMBUS Select by Yourgene Health Canada Inc.

Manual methods of recovering DNA from an electrophoretic gel include: (1) cutting out a slice of the gel corresponding to a band of DNA molecules of a particular length, followed by extraction of DNA from the gel slice and precipitation of DNA; and (2) collecting an aliquot or series of aliquots directly from the electrophoretic gel at a point in the gel that is accessible to a pipette, known in the art as an “extraction well”, followed by precipitation of DNA.

An automated electrophoresis system that can be used for size selection is described in U.S. Pat. No. 10,775,344 owned by Yourgene Health Canada Inc. and which is hereby incorporated by reference in its entirety. The electrophoresis system can be configured to distribute power to each gel channel and can modulate said power based upon a monitoring the current feedback on each channel. In an embodiment the system comprises an interfaced cassette comprising a plurality of channels, the system comprising: a robotic workstation for receiving at least one control signal from an external computing device for controlling electrophoresis operation thereon, the robotic workstation comprising: an arm comprising at least one pipette; and an on-board power module; a first modular pedestal electrically coupled and received on the robotic workstation, the interfaced cassette being housed within or positioned on the pedestal, the interfaced cassette comprising the plurality of channels, a pair of electrical contacts being associated with each of the plurality of channels, the first modular pedestal including a plurality of electrical conducting cables or wires to distribute an independently controllable power signal from the power module to each channel, the pedestal comprising a processor and a memory, the processor configured to: receive a power signal from the power module; receive said at least one control signal designated for the interfaced cassette of the first modular pedestal; and modulate the power signal in dependence upon the control signal to generate the independently controllable power signal defined for each channel of the interfaced cassette of the first modular pedestal. In an embodiment, the processor is configured to modulate the power signal further in dependence upon electrophoresis parameters stored on the memory and associated with each said cassette channel. The system can further comprise at least a second modular pedestal and a power transfer module electrically coupled to the power module, the power transfer module configured for communicating the power signal between the power module and said first modular pedestal and said at least second modular pedestal as a broadcast. The control signal is uniquely identifiable for each said cassette channel by multiplexing. Modulating the power signal can comprise modulating using at least one of: analog voltage control; pulse width modulation; duty cycle control for pulse width modulation; and frequency control for pulse width modulation. In particular embodiments, the processor is further configured to (i.) monitor a current feedback of each said channel; and (ii.) adjust the modulated power signal for each said channel in dependence upon said monitoring.

Other electrophoresis techniques are described as follows: capillary electrophoresis in a self-coating, low-viscosity polymer matrix is described in Du M, Flanagan J H Jr, Lin B, Ma Y, Electrophoresis 2003 September; 24 (18): 3147-53), which is hereby incorporated by reference in its entirety; selective extraction in a microfabricated electrophoresis device is described in Lin R, Burke D T, Burn M A, J. Chromatogr. A. 2003 Aug. 29; 1010(2): 255-68, which is hereby incorporated by reference in its entirety; and microchip electrophoresis on a reduced viscosity polymer matrices is described in Xu F, Jabasini M, Liu S, Baba Y, Analyst. 2003 June; 128(6): 589-92), which is hereby incorporated by reference in its entirety.

The size selection of cfDNA can also be done by chromatography such as chromatography on agarose or polyacrylamide gels, ion-pair reversed-phase high performance liquid chromatography (IP RP HPLC, see Hecker K H, Green S M, Kobayashi K, J. Biochem. Biophys. Methods 2000 Nov. 20; 46(1-2): 83-93, which is hereby incorporated by reference in its entirety), adsorptive membrane chromatography (see Teeters M A, Conrardy S E, Thomas B L, Root T W, Lightfoot E N, J. Chromatogr. A. 2003 Mar. 7; 989(1): 165-73 which is hereby incorporated by reference in its entirety); density gradient centrifugation (see Raptis L, Menard H A, J. Clin. Invest. 1980 December; 66(6): 1391-9, which is hereby incorporated by reference in its entirety); and methods utilizing nanotechnological means such as microfabricated entropic trap arrays (see Han J, Craighead H G, Analytical Chemistry, Vol. 74, No. 2, Jan. 15, 2002, which is hereby incorporated by reference in its entirety). A technique for bead-based size selection for cfDNA that can also be used is described in Raymond et al., UltraPrep is a scalable, cost-effective, bead-based method for purifying cell-free DNA. PLoS ONE 15(6) (2020): e0231854. https://doi.org/10.1371/journal.pone.0231854, which is hereby incorporated by reference in its entirety.

If the size selection step is performed after sequence library preparation, the desired cfDNA length maximum (e.g., less than 160 bp) or range of lengths is the same, but for the purpose of isolating the appropriate band coming off of the size selection device, the target maximum or range to be isolated would have to be adjusted to account for the adapters or other polynucleotides ligated on or inserted into the cfDNA. For example, if the use of adapters adds 100 bp to the length of a cfDNA polynucleotide and the target maximum was less than 160 bp, then the size selection parameters would be less than 260 bp.

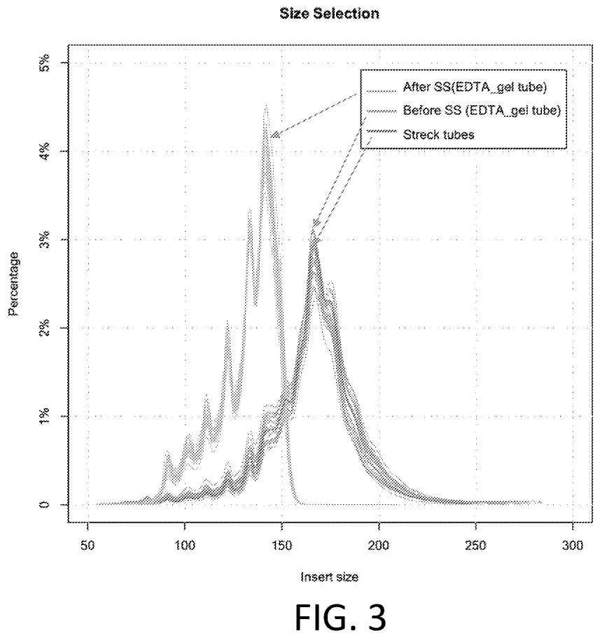

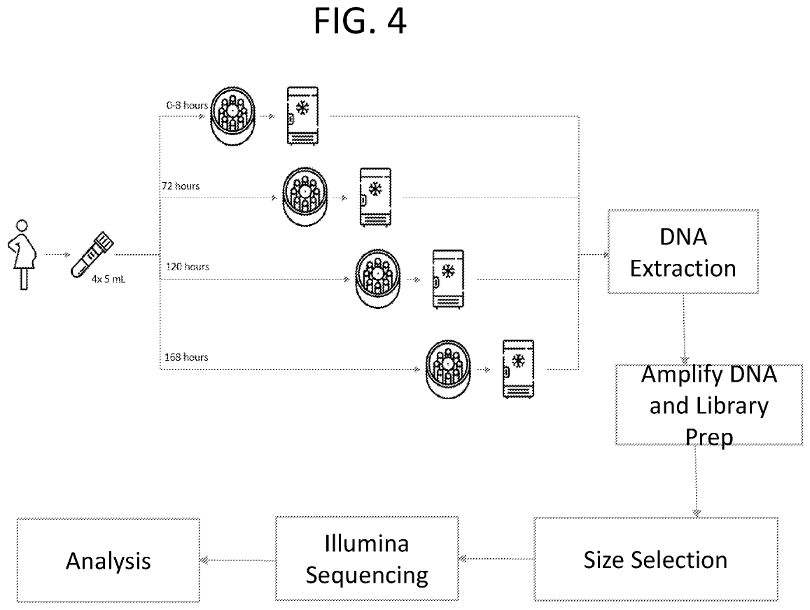

By performing the size selection step on a sample from a pregnant female, the percentage of fetal cfDNA in the sample to be analyzed for sequence information increases by 30%, 40%, 50%, 60%, 70%, 80%, 90%, 100%, 110%, 120%, 130%, 140%, 150%, 160%, 170%, 180%, 190%, or 200% compared to that before size selection. As shown in Example 1 and FIG. 3, the percentage of fetal cfDNA as estimated by SeqFF is about the same as or greater than the percentage of fetal cfDNA in a sample stored in Streck® tube for the same period. (SeqFF is the fetal fraction expressed as percentage of total cfDNA sequenced, calculated from sequence read counts. See Kim S K, et al.: Determination of fetal DNA fraction from the plasma of pregnant women using sequence read counts. Prenat Diagn 2015, 35:810-815, which is hereby incorporated by reference in its entirety.) Similarly, when looking at male only samples, the size selected sample has a greater percentage of cfDNA for all samples.

Similarly, by performing the size selection step on a sample for a cancer diagnostic, the percentage of cfDNA from cancer cells in the sample to be analyzed for sequence information increases by 30%, 40%, 50%, 60%, 70%, 80%, 90%, 100%, 110%, 120%, 130%, 140%, 150%, 160%, 170%, 180%, 190%, or 200% compared to that before size selection.

5.2.7 Analyzing the cfDNA to Obtain Sequence Information

Once size selection and library preparation is complete, the sample can be analyzed to obtain sequence information. Sequence information can be analyzed to detect mutations and chromosomal abnormalities including but not limited to translocation, transversion, monosomy, trisomy, and other aneuploidies, deletion, insertion, methylation, amplification, fragment, translocation, and rearrangement or chromathripsis. Sequence information can also be used to detect any alteration in gene sequence as compared to a reference sequence.

In an embodiment, the sample is sequenced as part of a procedure for determining a sequence of interest and for evaluating copy number variation(s) (CNVs). Any of a number of sequencing technologies can be utilized, including “first generation” sequencing, next generation sequencing, long-read sequencing, or nanopore sequencing.

Some sequencing technologies are available commercially, such as the sequencing-by-hybridization platform from Affymetrix Inc. (Sunnyvale, Calif.), the sequencing-by-synthesis platforms from Illumina, Inc. (Hayward, Calif.), and nanoball-based sequencing by Complete Genomics (San Jose, CA) Other single molecule sequencing technologies include, but are not limited to, the SMRT™ technology of Pacific Biosciences (see Anthony Rhoads et al., PacBio Sequencing and Its Applications, Genomics, Proteomics & Bioinformatics, Volume 13, Issue 5, 2015, page 278-289, ISSN 1672-0229, https://doi.org/10.1016/j.gpb.2015.08.002, which is hereby incorporated by reference in its entirety), the ION TORRENT™ technology, and nanopore sequencing developed for example, by Oxford Nanopore Technologies (see. Soni G V and Meller A. Clin Chem 53: 1996-2001 [2007], which is hereby incorporated by reference in its entirety).

While the automated Sanger method is considered as a ‘first generation’ technology, Sanger sequencing including the automated Sanger sequencing, can also be employed in the methods described herein. Additional suitable sequencing methods include, but are not limited to nucleic acid imaging technologies, e.g., atomic force microscopy (AFM) or transmission electron microscopy (TEM).

By way of example, Illumina's sequencing technology can be used to sequence the cfDNA. Illumina's technique relies on the attachment of cfDNA to a planar, optically transparent surface on which oligonucleotide anchors are bound. Template DNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchors. Under limiting-dilution conditions, adapter-modified, single-stranded template DNA is added to the flow cell and immobilized by hybridization to the anchors. Attached DNA fragments are extended and bridge amplified to create an ultra-high density sequencing flow cell with hundreds of millions of clusters, each containing 1,000 copies of the same template. In one embodiment, the cfDNA, is amplified using PCR before it is subjected to cluster amplification. Alternatively, an amplification-free genomic library preparation is used, and the cfDNA is enriched using the cluster amplification alone (Kozarewa et al., Nature Methods 6:291-295 (2009)). The templates are sequenced using a robust four-color DNA sequencing-by-synthesis technology that employs reversible terminators with removable fluorescent dyes. High-sensitivity fluorescence detection is achieved using laser excitation and total internal reflection optics. Short sequence reads of about 20-300 bp, e.g., 150 bp, can be aligned against a repeat-masked reference genome and unique mapping of the short sequence reads to the reference genome are identified using specially developed data analysis pipeline software. Non-repeat-masked reference genomes can also be used. Whether repeat-masked or non-repeat-masked reference genomes are used, only reads that map uniquely to the reference genome are counted. After completion of the first read, the templates can be regenerated in situ to enable a second read from the opposite end of the fragments. Thus, either single-end or paired end sequencing of the DNA fragments can be used. Partial sequencing of DNA fragments present in the sample is performed, and sequence tags comprising reads of predetermined length, e.g., 36 bp, are mapped to a known reference genome are counted. In one embodiment, the reference genome sequence is the NCBI36/hg18 sequence, which is available on the world wide web at genome.ucsc.edu/cgi-bin/hgGateway?org-Human&db=hg18&hgsid=166260105). Alternatively, the reference genome sequence is the GRCh37/hg19, which is available on the world wide web at genome.ucsc.edu/cgi-bin/hgGateway. Other sources of public sequence information include GenBank, dbEST, dbSTS, EMBL (the European Molecular Biology Laboratory), and the DDBJ (the DNA Databank of Japan). A number of computer algorithms are available for aligning sequences, including without limitation BLAST (Altschul et al., 1990), BLITZ (MPsrch) (Sturrock & Collins, 1993), FASTA (Person & Lipman, 1988), or BOWTIE (Langmead et al., Genome Biology 10:R25.1-R25.10 (2009)), all of which are hereby incorporated by reference in their entirety.

By way of another example, sequence information for the nucleic acids in the test sample, e.g., cfDNA in a maternal test sample or cfDNA in a subject being screened for a cancer, can be obtained using the 454 sequencing (Roche) (e.g. as described in Margulies, M. et al Nature 437:376-380 [2005], which is hereby incorporated by reference in their entirety). 454 sequencing typically involves two steps. In the first step, DNA is sheared into fragments of approximately 300-800 base pairs, and the fragments are blunt-ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (e.g., picoliter-sized wells). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is measured and analyzed.

In another illustrative, but non-limiting, embodiment, sequence information can be obtained by performing a sequencing-by-ligation, which shears genomic DNA into fragments, and attaches adaptors to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

In another embodiment, sequence information for the nucleic acids in the test sample, e.g., cfDNA in a maternal test sample, cfDNA in a subject being screened for a cancer, can be obtained using the single molecule, real-time (SMRT™) sequencing technology of Pacific Biosciences. In SMRT sequencing, the continuous incorporation of dye-labeled nucleotides is imaged during DNA synthesis. Single DNA polymerase molecules are attached to the bottom surface of individual zero-mode wavelength detectors (ZMW detectors) that obtain sequence information while phospholinked nucleotides are being incorporated into the growing primer strand. A ZMW detector comprises a confinement structure that enables observation of incorporation of a single nucleotide by DNA polymerase against a background of fluorescent nucleotides that rapidly diffuse in an out of the ZMW (e.g., in microseconds). It typically takes several milliseconds to incorporate a nucleotide into a growing strand. During this time, the fluorescent label is excited and produces a fluorescent signal, and the fluorescent tag is cleaved off. Measurement of the corresponding fluorescence of the dye indicates which base was incorporated. The process is repeated to provide a sequence.

In another embodiment, sequence information for the nucleic acids in the test sample, e.g., cfDNA in a maternal test sample or cfDNA in a subject being screened for a cancer, is obtained by using nanopore sequencing. Nanopore sequencing DNA analysis techniques are developed by a number of companies, including, for example, Oxford Nanopore Technologies (Oxford, United Kingdom), Sequenom, and NABsys. Nanopore sequencing is a single-molecule sequencing technology whereby a single molecule of DNA is sequenced directly as it passes through a nanopore. A nanopore is a small hole, typically of the order of 1 nanometer in diameter. Immersion of a nanopore in a conducting fluid and application of a potential (voltage) across it results in a slight electrical current due to conduction of ions through the nanopore. The amount of current that flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore provides a read of the DNA sequence.

In another embodiment, sequence information for the nucleic acids in the test sample, e.g., cfDNA in a maternal test sample or cfDNA in a subject being screened for a cancer, can be obtained by using the chemical-sensitive field effect transistor (chemFET) array (e.g., as described in US Patent Application Publication No. 2009/0026082, which are hereby incorporated by reference in their entirety). In one example of this technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned as a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In another embodiment, sequence information is obtained using the Ion Torrent single molecule sequencing, which pairs semiconductor technology with a simple sequencing chemistry to directly translate chemically encoded information (A, C, G, T) into digital information (0, 1) on a semiconductor chip. In nature, when a nucleotide is incorporated into a strand of DNA by a polymerase, a hydrogen ion is released as a byproduct. Ion Torrent uses a high-density array of micro-machined wells to perform this biochemical process in a massively parallel way. Each well holds a different DNA molecule. Beneath the wells is an ion-sensitive layer and beneath that an ion sensor. When a nucleotide, for example a C, is added to a DNA template and is then incorporated into a strand of DNA, a hydrogen ion will be released. The charge from that ion will change the pH of the solution, which can be detected by Ion Torrent's ion sensor. The sequencer—essentially the world's smallest solid-state pH meter—calls the base, going directly from chemical information to digital information. The Ion personal Genome Machine (PGM™) sequencer then sequentially floods the chip with one nucleotide after another. If the next nucleotide that floods the chip is not a match. No voltage change will be recorded and no base will be called. If there are two identical bases on the DNA strand, the voltage will be double, and the chip will record two identical bases called. Direct detection allows recordation of nucleotide incorporation in seconds.

In another embodiment, the present method comprises obtaining sequence information for the nucleic acids in the test sample, e.g., cfDNA in a maternal test sample, using sequencing by hybridization. Sequencing-by-hybridization comprises contacting the plurality of polynucleotide sequences with a plurality of polynucleotide probes, wherein each of the plurality of polynucleotide probes can be optionally tethered to a substrate. The substrate might be flat surface comprising an array of known nucleotide sequences. The pattern of hybridization to the array can be used to determine the polynucleotide sequences present in the sample. In other embodiments, each probe is tethered to a bead, e.g., a magnetic bead or the like. Hybridization to the beads can be determined and used to identify the plurality of polynucleotide sequences within the sample.

In another embodiment, the present method comprises obtaining sequence information for the nucleic acids in the test sample, e.g., cfDNA in a maternal test sample, by massively parallel sequencing of millions of DNA fragments using Illumina's sequencing-by-synthesis and reversible terminator-based sequencing chemistry (e.g. as described in Bentley et al., Nature 6:53-59 [2009], which is hereby incorporated by reference in its entirety).

Mapping of the sequence tags is achieved by comparing the sequence of the tag with the sequence of the reference to determine the chromosomal origin of the sequenced nucleic acid molecule (in this case, cfDNA), and specific genetic sequence information is not needed. A small degree of mismatch (0-2 mismatches per sequence tag) may be allowed to account for minor polymorphisms that may exist between the reference genome and the genomes in the mixed sample.

A plurality of sequence tags are typically obtained per sample. The sequence tags comprise between 20 and 185 bp reads, e.g., 100 bp. In one embodiment, all the sequence reads are mapped to all regions of the reference genome. In one embodiment, the tags that have been mapped to all regions, e.g., all chromosomes, of the reference genome are counted, and the CNV, i.e., the over- or under-representation of a sequence of interest, e.g., a chromosome or portion thereof, in the mixed DNA sample is determined. The method does not require differentiation between the two genomes.

The accuracy required for correctly determining whether a CNV, e.g., aneuploidy, polyploidy, and deletion, is present or absent in a sample, is predicated on the variation of the number of sequence tags that map to the reference genome among samples within a sequencing run (inter-chromosomal variability), and the variation of the number of sequence tags that map to the reference genome in different sequencing runs (inter-sequencing variability). For example, the variations can be particularly pronounced for tags that map to GC-rich or GC-poor reference sequences. Other variations can result from using different protocols for the extraction and purification of the nucleic acids, the preparation of the sequencing libraries, and the use of different sequencing platforms. The present method uses sequence doses (chromosome doses, or segment doses) based on the knowledge of normalizing sequences (normalizing chromosome sequences or normalizing segment sequences), to intrinsically account for the accrued variability stemming from interchromosomal (intra-run), and inter-sequencing (inter-run) and platform-dependent variability. Chromosome doses are based on the knowledge of a normalizing chromosome sequence, which can be composed of a single chromosome, or of two or more chromosomes selected from chromosomes 1-22, X, and Y. Alternatively, normalizing chromosome sequences can be composed of a single chromosome segment, or of two or more segments of one chromosome or of two or more chromosomes. Segment doses are based on the knowledge of a normalizing segment sequence, which can be composed of a single segment of any one chromosome, or of two or more segments of any two or more of chromosomes 1-22, X, and Y.

In an embodiment, the cfDNA is sequenced to determine the sequence of alleles of a locus of interest within the sample. From the sequences, a heterozygous locus of interest is identified and a ratio of the alleles is quantified. The ratio indicates the presence or absence of a chromosomal abnormality.

In some embodiments, the methods also involve determining whether the fetus has a genetic disease from the at least one sequence of interest of the fetus. In some embodiments, this includes determining whether the fetus is homozygous in a disease causing allele within the sequence of interest when the mother is heterozygous of the same allele. In some embodiments, the disease causing allele is an allele of a single nucleotide polymorphism (SNP), in the sequence of interest. In some embodiments, the disease causing allele is an allele of a short tandem repeat (STR) in the sequence of interest.

In some embodiments, the at least one sequence of interest comprises a site of an allele associated with a disease. The at least one sequence of interest may include one or more of the following: single nucleotide polymorphism, tandem repeat, micro-deletion, insertion, and indel. In some embodiments, the methods further involve determining if the fetus is homozygous or heterozygous for the disease associated allele.

In some embodiments, the method further involves counting the sequence tags to determine a copy number variation (CNV) of a chromosomal sequence, and/or examining sequences of the sequence tags to detect non-copy number variations of a chromosomal sequence. Some embodiments further involve determining for the fetus the presence or absence of a polymorphism or chromosomal abnormality. In some embodiments, the method further involves determining for the fetus the presence or absence of a SNP, tandem repeat, insertion, deletion, indel, translocation, duplication, inversion, CNV, partial aneuploidy, and/or complete aneuploidy. The complete chromosomal aneuploidy may be a duplication, a multiplication, or a loss of a complete chromosome. In some embodiments, the method further involves localizing the partial aneuploidy. In some embodiments, the SNP is selected from a single SNP and a tandem SNP. In some embodiments, the tandem repeat is selected from a dinucleotide repeat and a trinucleotide repeat.

In some embodiments, the method further involves determining a normalized chromosome sequence value for a chromosome sequence of interest and comparing the sequence value to an upper and a lower threshold value, wherein the sequence value exceeding the upper or the lower threshold values respectively determines the presence or absence of the CNV.

In some embodiments, analyzing the plurality of sequence tags involves determining the presence or absence of a chromosomal abnormality of chromosomes 1-22, X and Y. Some embodiments further involve determining whether the fetus is homozygous in an allele within the sequence of interest, or determining that the mother is heterozygous and the fetus is homozygous. Some embodiments further involve determining the fetal fraction of the cfDNA.

Methods and systems for analyzing cfDNA is described in U.S. Pat. No. 10,837,055, which is hereby incorporated by reference in its entirety.

Another aspect of this disclosure is a sequence validity test that can detect for abnormal performance of the size selection step or unacceptable sample degradation. An embodiment of this test is described in Example 4 and was used to evaluate the suitability of a sample in Example 3. While Example 3 describes use of the method in the context of using with a K2EDTA tube, it is understood that this validity test can be used to evaluate the integrity of a sample where a size selection step is performed, regardless of collection tube type or storage time or conditions. In a method involving size selection of a DNA sample comprising cfDNA and the sequencing of the sample DNA after size selection; the method can comprise a sequence validity test of sequence information obtained from the sequencing step. The validity test comprises creating a first normalised fragment size profile from the sequencing information associated with the sample, comparing one or more values within the fragment size profile against one or more of corresponding value ranges obtained from a reference parameter set comprising a plurality of valid fragment size profiles; and accepting the sequence information and the analysis results thereof if one of the one or more values within the fragment size profile associated with the sample is within the corresponding value range or rejecting the sequence information if one of the one or more values within the fragment size profile associated sample is outside of the corresponding value range. The validity test may also comprise calculating a first relative fragment size frequency value from the sequencing information associated with the sample; comparing the first relative fragment size frequency value to a reference relative fragment size frequency value set comprising a plurality of valid relative fragment size frequencies, and accepting the sequence information and the analysis results thereof if the first fragment size frequency associated with the sample is within a specified range of values within the reference set or rejecting the sequence information and the analysis results thereof if the first fragment size frequency associated with the sample is outside a specified range of values within the reference set.

### EXAMPLES

### 5.3. Example 1

This study is a comparative study evaluating the quality of cfDNA retrieved from blood stored and transported in either K₂EDTA tube with gel (after low centrifugation) and uncentrifuged Streck tube. Previous work has shown that EDTA-gel tube was as good as Streck tube to collect blood and prepare cfDNA for NIPT without reduction of fetal fraction if blood was processed the same day (Giroux, 2021). The present study tests the possibility to keep the centrifuged tube at 4° C. for five days and ship it cold to the sequencing laboratory at 4800 km away.

Pregnant women were exclusively recruited in Vancouver after consenting to participate to the study. All the pregnant women recruited were between 10 and 14 weeks of maternal age. A details of the study population is shown in Table 1.

TABLE 1

| | |
|---|---|
| Number of pregnant women | 61 |
| Maternal age (weeks) | |
| Mean | 10.9 |
| range | 10-13.9 |
| Weight (kg) | |
| Mean | 63.9 |
| range | 41.3-136 |
| Body Mass Index | |
| Mean | 23.8 |
| range | 17-42 |
| Number of women with weight >80 kg | 5 |
| Number of women with BMI >30 | 5 |

For each participant, two 10 ml peripheral blood samples were collected in Streck tubes Cat #218997 (Streck, NB, USA) and 8 ml was collected in K2EDTA Gel blood tubes for molecular diagnostics Cat #GR-455040 (Greiner, Austria). The EDTA-gel tubes were centrifuged at 1800 g for 10 min within 6 hours of sample collection (as recommended by the manufacturer) and then kept at 4° C. until shipped cold once a week to a laboratory in Quebec City. Blood collected in Streck tubes were shipped at room temperature twice a week to the same laboratory. In total, 61 samples collected in Vancouver and shipped over 4800 km to a testing laboratory in Quebec City

Plasma was collected carefully using a 1 ml transfer pipet from Streck tubes 2 to 5 days after blood collection. Plasma was filtered through a 0.45 μM HPF-Millex-PVDF-Durapore from Millipore (Merck, MA, USA) using a 5 ml syringe. Plasma in the K₂EDTA tube was decanted into the 5 ml syringe cylinder and filtered using the same type of filter as above as previously reported (Giroux, 2021). EDTA-gel tubes spent 2 to 9 days between blood collection and filtration.

cfDNA was prepared from 2 to 5 ml plasma using the QIAamp Circulating Nucleic Acid kit Cat #55114 (Qiagen, Germany). DNA was recovered in 50 or 70 μl and quantified by fluorometry using the Qubit dsDNA HS Assay Kit Cat #Q32851 (Thermofisher, MA, USA). Volumes of 15 to 25 μl containing between 2 and 15 ng of cfDNA were used to prepare libraries using the KAPA Hyper Prep kit Cat #07962363001 (Roche, Switzerland) following the manufacturer's instructions with some modifications. Volumes were reduced by half at every step. Kapa Unique Dual-indexed adapters Cat #08861919702 (Roche, Switzerland) were used at 0.5 μM final concentration. Ten cycles of amplification were performed and a post-amplification clean-up with 0.6×-1× Kapa beads Cat #07983298001 (Roche, Switzerland) was performed.

For one group of samples, libraries were quantified by fluorometry using the Qubit dsDNA HS Assay Kit as above. Molarity was calculated using an average size of 325 base pairs (bp). Pool of up to 17 equimolar libraries were prepared to sequence paired-ends 150 cycles on the Illumina Next-Seq 550 instrument with a mid-output flow cell Cat #20024909 (Illumina, CA, USA).

For a second group of samples, libraries were size-selected using the LightBench from Yourgene Health Canada Inc. (Manchester, UK). 25 μl sample plus loading buffer and 200 bp/300 bp labeled markers were run together on a 3% precast gel. Software was programmed to extract DNA ranging from 229 bp to 286 bp. DNA recovered was quantified with Qubit and mass was converted to molarity using an average size of 275 bp. Pool of up to 25 libraries were prepared and sequenced on mid-output flow cell as above.

### Results

Using the standard procedure with Streck tube, the samples arrived within 5 days at room temperature. Seven women had a fetal fraction lower than 4% and six of them had a weight greater than 70 kg or a BMI greater than 25.

With the EDTA-gel tubes, samples arrived also within 5 days but had to remain cold. Most of the samples arrived within two days with a Priority Shipping with the chosen transporter. However, two shipments involving 14 samples were delayed 4 and 5 days, and these samples were no longer kept cold. These 14 samples suffered from their transportation and had a much lower fetal fraction than expected. Their average fetal fraction reduction was 4.6% compared to that obtained with Streck tube. The EDTA-gel tubes that arrived cold in two days or less (N=47) had a similar fetal fraction to the one observed with Streck tube but reduced on average by 1.1%. There was a trend between the time spent at 4° C. (tubes were kept in the refrigerator until shipped) and the extent of the fetal fraction reduction. We observed an increased fetal fraction reduction with increased time before treatment from 0.75% reduction for 2-3 days to 1.41% reduction for 6 days or more.

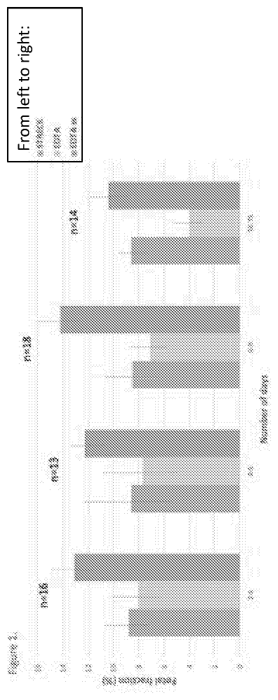

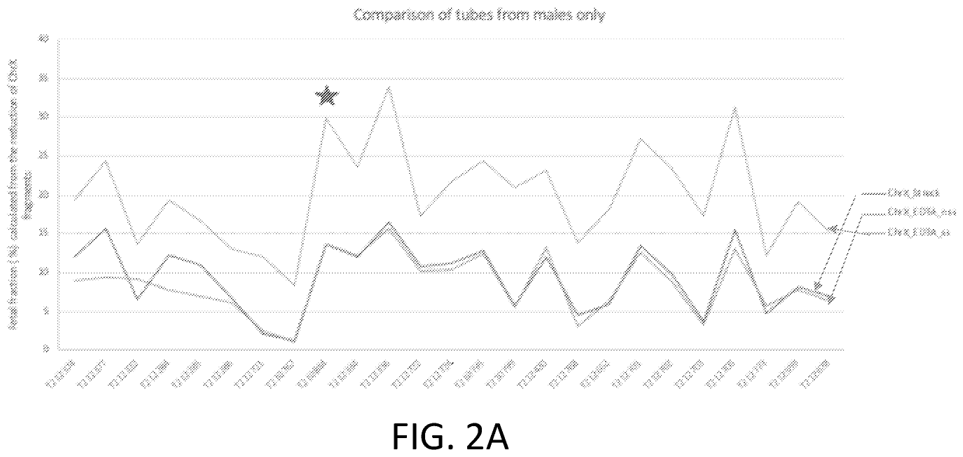

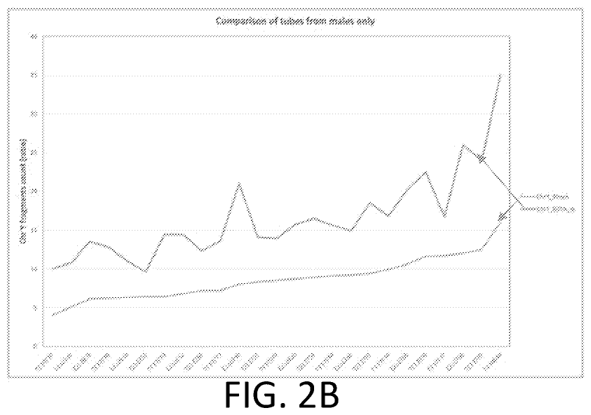

All libraries prepared from DNA isolated from plasma from EDTA-gel tubes were run on a 3% gel electrophoresis using the LightBench™ instrument from Yourgene Health Canada Inc. Software was set to collect fragments 229 base pairs to 286 base pairs, which accounts for adaptors adding 136 bp to a cfDNA strand. The material collected was sequenced and fetal fraction was evaluated with SeqFF as usual. With the exception of 5 samples, a fetal fraction increase was observed. Those 5 which did not exhibit an increase had higher fetal fractions initially. Samples delayed in transport for five days were those who increased less compared to Streck tube results. Two had a fetal fraction smaller by 0.4% and 3%, respectively, compared to Streck tube but the fetal fraction was improved compared to the EDTA-gel tube before size-selection and the twelve others increased by 0.1 to 1.5% (FIG. 1. Samples in the left). The 47 remaining samples all increased on average by 5.4% ranging from 0.8 to 10.2% except for 3 samples remaining above 10% but decreased compared to Streck and even EDTA-gel before size-selection (FIG. 1. Samples with stars). Determining the fetal fraction with SeqFF yields a rough estimate but is nevertheless very useful for female pregnancy. Also determined was the fetal fraction calculated from the reduction of ChrX fragments in male pregnancy and found an important increase for each sample analyzed (FIG. 2A). Even a sample with lower fetal fraction with SeqFF appeared much higher with this method to calculate (blue star in FIGS. 1 and 2A). The tube used and the selection on 3% agarose did not artifactually increase the fetal fraction by interfering with SeqFF counting, as an increase in reads from chromosome Y in pregnancies with male fetus (n=25) was also observed. (Supp FIG. 2B).

FIG. 3 shows the size of the sequenced fragments calculated from the mapping of paired-ends sequencing. In grey fragments before the size-selection and in blue after size-selection.

TABLE 2

| | Streck | EDTA-ALL | EDTA-4° C. | EDTA + SS |
|---|---|---|---|---|
| Mean FF (%) | 8.6 | 6.8 | 7.6 | 12.6 |
| FF Range (%) | 2.3-26.8 | 0.4-25.2 | 0.4-25.2 | 6.4-22.6 |
| No. of samples | 7 | 16 | 9 | 0 |
| w/FF <4% | | | | |

### Example 2

The objective of this second study is to assess the impact of extending the storage time before centrifugation of whole blood collected in K₂EDTA tube on the detection of fetal cfDNA. Separate tubes from a single blood draw from each participant will be incubated for a range of time points, centrifuged to obtain plasma, then processed to isolate and amplify the DNA, perform a size selection, create a DNA library, and sequence the cfDNA. Fetal fraction percentage and chromosome ratios will be used to determine the effect of storage time prior to centrifugation. This will enable measuring the impact of variable blood storage time on fetal fraction percentage.

Samples will be processed within at the following time points: Time point 1—0-8 hrs, Time point 2—72 hrs, Time point 3—120 hrs, and Time point 4—168 hrs. During this time, a portion of samples will be stored at refrigeration temperatures and a portion will be stored at room temperature. At the appropriate time point, plasma will be separated from the whole blood via centrifugation. All tubes were centrifuged at 1600 g×10 min within 6 h of sample collection. Plasma from EDTA tubes was poured into a syringe cylinder and filtered through a 0.45 μm Millipore filter. Isolated plasma samples will be stored at −80° C. until at least ninety-six samples are collected, at which point DNA extraction will commence. Storage at this temperature will preserve the sample until extraction. Extraction of the cfDNA can be performed with the QIAamp Circulating Nucleic Acid kit Cat #55114 (Qiagen, Germany). A LightBench® by Yourgene Health Canada Inc. will be used to select for a certain size of cfDNA in the sample. The size selection parameters are the same as in Example 1. NGS sequencing libraries were prepared and sequenced on an Illumina system. Fetal fractions were estimated using SeqFF.

### Example 3

The objective of this third study was also to assess the impact of extending the storage time before centrifugation of whole blood collected in K₂EDTA blood tube (aka K₂EDTA vacutainers) on the detection of fetal cfDNA. Separate tubes from a single blood draw from each participant were incubated for Time Points 1-4, centrifuged twice to obtain plasma, then processed to isolate and amplify the DNA, create a DNA sequencing library, pool the sequencing libraries, carry out size selection and sequence the cfDNA. Fetal fraction percentage and chromosome ratios were used to determine the effect of storage time prior to centrifugation. This enabled measuring the impact of variable blood storage time on fetal fraction percentage and the quality of NIPT testing in standard blood tubes with delayed plasma isolation.

Samples were stored at ambient temperatures and processed within at the following time points: Time point 1—0-8 hrs, Time point 2—72 hrs, Time point 3—120 hrs, and Time point 4—168 hrs. Ambient temperatures ranged from 20 C to 35 C. At the appropriate time point, plasma was separated from the whole blood via double centrifugation. The plasma isolation centrifugation process comprises a first centrifuging step for 10 minutes at 1600 rpm and a second centrifuging step for 10 minutes at 3000 rpm. After the first centrifuging, plasma from the K2EDTA tube was transferred to a second tube and subject to the second centrifuging step. After the second step, plasma was removed from the second tube and transferred to a third tube and stored at −80° C. until enough samples were collected to commence </=48 sample run, at which point DNA extraction commenced. Isolated plasma samples were stored at −80° C. until about 48 samples were collected, at which point the process of DNA extraction commenced. Storage at this temperature preserved the sample until extraction.

Extraction of the cfDNA was performed with the MagMAX™ Cell-Free DNA Isolation Kit. Despite not using Streck tubes, the lysis step was carried out as this part of the script and has no detrimental effect on samples collected in EDTA tubes. A QS250™ by Yourgene Health Canada Inc. was used to select for a certain size of cfDNA in the sample. The size selection parameters were 106-146 bp (value does not include adapter length) and recovered range was ˜100-186 bp (mean fragment length is ˜143-146 bp). NGS sequencing libraries were prepared and sequenced on an Illumina system. Fetal fractions were estimated using SeqFF

### Sample Set Details

105 matched sets of clinical samples were collected for this Study. Samples were collected with a known gestational age, height, weight and BMI. Separate vacutainers from a single blood draw from each participant were incubated Time Points 1-4, centrifuged to obtain plasma, then run through the IONA® Nx workflow. Fetal fraction percentage and chromosome ratios were used to determine the effect of storage time prior to centrifugation.

From nearly all participants, four—5 mL blood specimens (termed matched specimens) were collected from each participant in BD Vacutainer™ Plastic K₂EDTA Tubes from one hundred and five patient volunteers (pregnant women>10+0 weeks' gestation). For each participant, matched 5 mL aliquots underwent centrifugation at each of the Time Points 1 to 4 after incubation at ambient temperature. The following Table 3 provides a summary of samples that were collected. Those time points deviating from 105 samples are less than 105 samples due to obtaining less than 20 mL from the participant.

TABLE 3

| Total No. of Patients | | 77 | | |
|---|---|---|---|---|
| Sample No. | Sample No. | Sample No. | Sample No. | Total Sample |
| Time Point 1 | Time Point 2 | Time Point 3 | Time Point 4 | No. |
| 77 | 77 | 75 | 73 | 302 |

Participants were designated as M029 to M105. The following samples mistakenly underwent a single centrifugation instead of double centrifugation as is standard in the IONA Nx protocol:
M029-M30 Time Points 1-3
M031-M032 Time Points 1-2
   These samples have been excluded from this report due to the single spin method of plasma preparation used.

The remaining samples underwent double centrifuging, which are summarized below
M029-M030 Time Point 4
M031-M032 Time Point 3 and 4
M033-M105 Time Point 1,2,3 and 4
   The above samples are those that were included in the analysis.

### Participant Exclusion and Inclusion Criteria

Inclusion and exclusion criteria were as follows:

### Inclusion Criteria

Pregnant woman≥18 years old
Women who were able to understand and provide informed consent
Singleton pregnancy≥10+0 weeks' gestation with no upper limit cut off for gestation i.e., all the way to due date

### Exclusion Criteria

Known maternal aneuploidy or maternal cancer
Twin or vanishing twin cases
Known infectious disease e.g., current COVID-19 infection or within COVID-19 isolation period, Hep B
Anyone deemed to belong to a vulnerable group
Anyone known to have had an organ or stem cell transplant
Anyone known to have had a blood transfusion in last 12 months

### Results

### Summary of Samples

A QC validity status summary of all double spun, patient samples at the four time points used within this study is shown below in Table 4.

TABLE 4

| Patient Number | Time Point 1 | Time Point 2 | Time Point 3 | Time Point 4 |
|---|---|---|---|---|
| M029 | NA | NA | NA | Valid |
| M030 | NA | NA | NA | Valid |
| M031 | NA | NA | Valid | Valid |
| M032 | NA | NA | Valid | Valid |
| M033 | Valid | Valid | Valid | Valid |
| M034 | Valid | Valid | Valid | Valid |
| M035 | Valid | Valid | Valid | Valid |
| M036 | Valid | Valid | Valid | Valid |
| M037 | Valid | Valid | Valid | Valid |
| M038 | Valid | Valid | Valid | Valid |
| M039 | Valid | Valid | Valid | Valid |
| M040 | Failed LP Validity | Valid | Sample Unavailable | Valid |
| M041 | Valid | Valid | Valid | Valid |
| M042 | Valid | Valid | Valid | Valid |
| M043 | Valid | Valid | Valid | Valid |
| M044 | Valid | Valid | Valid | Valid |
| M045 | Valid | Valid | Valid | Valid |
| M046 | Valid | Valid | Valid | Valid |
| M047 | Valid | Valid | Valid | Valid |
| M048 | Valid | Valid | Valid | Valid |
| M049 | Valid | Valid | Valid | Valid |
| M050 | Valid | Valid | Valid | Valid |
| M051 | Valid | Valid | Valid | Valid |
| M052 | Valid | Valid | Valid | Valid |
| M053 | Valid | Valid | Valid | Valid |
| M054 | Valid | Valid | Valid | Valid |
| M055 | Valid | Valid | Valid | Valid |
| M056 | Failed LP Validity* | Valid | Valid | Valid |
| M057 | Valid | Valid | Valid | Valid |
| M058 | Valid | Valid | Valid | Valid |
| M059 | Valid | Valid | Valid | Valid |
| M060 | Valid | Valid | Valid | Valid |
| M061 | Valid | Valid | Valid | Valid |
| M062 | Valid | Valid | Valid | Valid |
| M063 | Valid | Valid | Valid | Valid |
| M064 | Valid | Valid | Valid | Valid |
| M065 | Valid | Valid | Valid | Valid |
| M066 | Valid | Valid | Valid | Valid |
| M067 | Valid | Valid | Valid | Valid |
| M068 | Valid | Valid | Failed Sequencing | Valid |
| | | | Validity | |
| M069 | Valid | Valid | Valid | Valid |
| M070 | Valid | Valid | Valid | Valid |
| M071 | Valid | Valid | Valid | Sample Unavailable |
| M072 | Valid | Valid | Valid | Valid |
| M073 | Valid | Valid | Valid | Valid |
| M074 | Valid | Valid | Valid | Valid |
| M075 | Valid | Valid | Valid | Valid |
| M076 | Valid | Valid | Failed Sequencing | Valid |
| | | | Validity | |
| M077 | Valid | Valid | Valid | Valid |
| M078 | Valid | Valid | Valid | Valid |
| M079 | Valid | Sample Unavailable | Valid | Valid |
| M080 | Valid | Valid | Valid | Failed Sequencing |
| | | | | Validity |
| M081 | Valid | Valid | Valid | Sample Unavailable |
| M082 | Valid | Sample Unavailable | Valid | Valid |
| M083 | Valid | Valid | Valid | Valid |
| M084 | Sample Unavailable | Valid | Valid | Valid |
| M085 | Valid | Valid | Valid | Failed Sequencing |
| | | | | Validity |
| M086 | Valid | Valid | Valid | Valid |
| M087 | Valid | Valid | Valid | Valid |
| M088 | Omitted | Omitted | Omitted | Omitted |
| M089 | Valid | Failed LP Validity | Valid | Failed Sequencing |
| | | | | Validity |
| M090 | Failed LP Validity | Valid | Valid | Valid |
| M091 | Valid | Valid | Valid | Valid |
| M092 | Failed LP Validity | Failed LP Validity | Valid | Valid |
| M093 | Valid | Valid | Valid | Valid |
| M094 | Valid | Valid | Valid | Valid |
| M095 | Valid | Valid | Valid | Valid |
| M096 | Valid | Valid | Valid | Valid |
| M097 | Valid | Valid | Valid | Valid |
| M098 | Valid | Valid | Valid | Valid |
| M099 | Failed LP Validity | Valid | Valid | Valid |
| M100 | Valid | Valid | Valid | Valid |
| M101 | Failed LP Validity | Valid | Failed Sequencing | Failed Sequencing |
| | | | Validity | Validity |
| M102 | Valid | Valid | Valid | Failed Sequencing |
| | | | | Validity |
| M103 | Valid | Valid | Valid | Valid |
| M104 | Failed LP Validity | Valid | Valid | Valid |
| M105 | Valid | Valid | Valid | Valid |

Table 1: Summary of all patient samples, time points and sample validity status, inclusive of 1x retest sample validity, of all double-spun samples used within this study.
LP = Library Preparation;
*= M056 (Time Point 1) was pooled and size selected in Run 5 despite being invalid as detailed in Section 8.4;
Sample Unavailable = Sample not received to process;
Omitted = Single spun samples;
Omitted for other reason.

### Library Validity

Library Preparation (:LP) validity rates were calculated for each run and for each respective time point, where libraries with a post-PCR quantification value of ≥2.5 ng/μL were considered valid.

The overall initial library validity and rate validity rate after 1× retest for all samples at each Time Point in the study is shown below in 5.

TABLE 5
Initial library validity and 1x retest rates for across each Time
Point and the overall validity rate as a part of this study.

| | Time Point | Time Point | Time Point | Time Point | |
|---|---|---|---|---|---|
| | 1 | 2 | 3 | 4 | Overall |
| Initial Valid | 60/70 | 65/68 | 71/72 | 74/74 | 270/284 |
| Libraries | | | | | |
| Initial | 85.71% | 95.59% | 98.61% | 100.00% | 95.07% |
| Validity | | | | | |
| Rate | | | | | |
| 1x Retest | 63/70 | 66/68 | 72/72 | — | 275/284 |
| Valid | | | | | |
| Libraries | | | | | |
| 1x Retest | 90.00% | 97.06% | 100.00% | 100.00% | 96.83% |
| Validity | | | | | |
| Rate | | | | | |

Time Point 1 = 0-8 hours, Time Point 2 = 72 hours, Time Point 3 = 120 hours and Time Point 4 = 168 hours

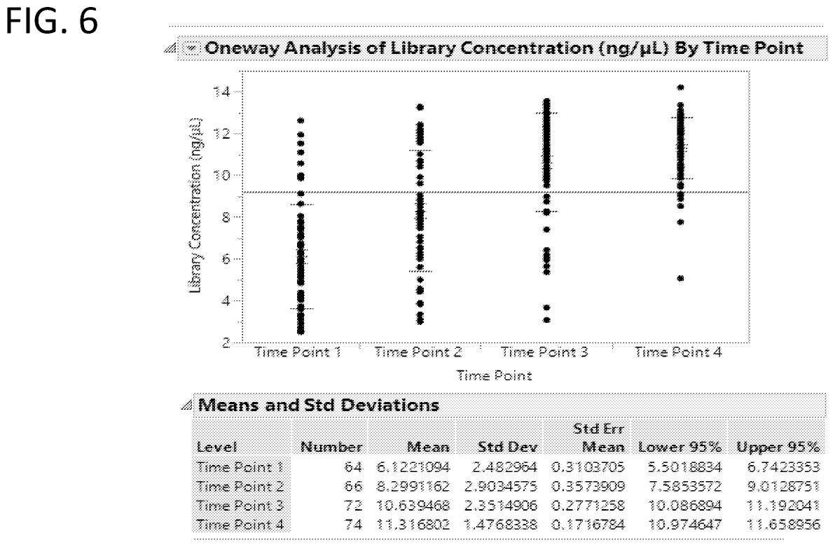

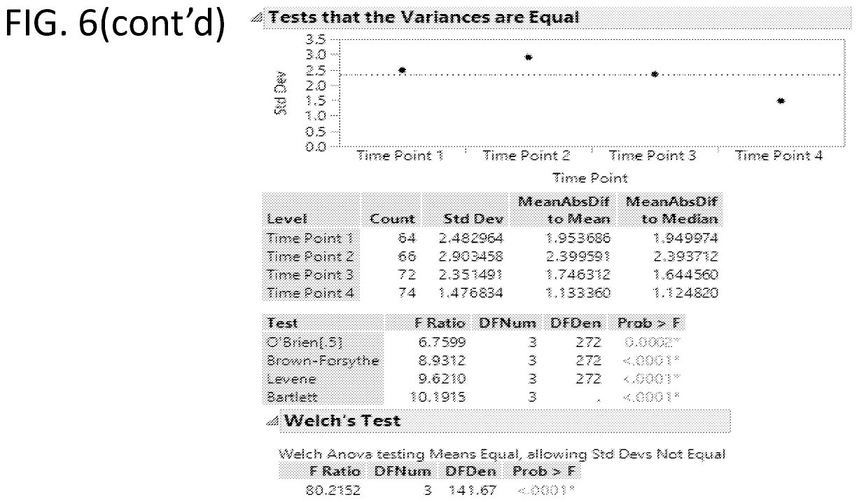

Summary statistics (means and standard deviations) and a Welch ANOVA test were performed for the 275 valid libraries after 1× retest (referenced in 5) plus the sample M056 (Time Point 1) from Run 5 that was included in the sample pool for size selection due to being close to meeting the library validity criteria (2.49 ng/μL). The data for all 276 samples is shown in FIG. 6.

A Welch ANOVA test was performed in place of ANOVA as the unequal variances test demonstrated that the standard deviations for the data from each timepoint were not equal (see FIG. 6); as such, ANOVA was not an appropriate analysis method as this test assumes equal variances.

Visually, the data demonstrates a shifted higher library concentration concomitant with extended sample storage time in K2EDTA tubes prior to centrifugation. Time Point 4 demonstrated the largest mean library concentration (11.31 ng/μL, FIG. 6 Error! Reference source not found.), whereas Time Point 1 demonstrated the lowest mean concentration (6.12 ng/μL, Figure Error! Reference source not found.6). Time Point 2 presented the largest standard deviation (SD=2.90, FIG. 6), with Time Point 4 presenting with the smallest (SD=1.48, Figure Error! Reference source not found.6). Time Point 1, Time Point 2 and Time Point 3 had a standard deviation of 2.48, 2.90 and 2.35 respectively, suggesting there is comparable variance across these Time Points. These observations are consistent with the hypothesis that extended storage time of samples in the EDTA tubes causes increased DNA input into the library preparation reaction; later Time Points would therefore be expected to return higher library concentrations.

The Welch ANOVA p-value of <0.0001 in 6 indicates there is a statistically significant variance in the library quantification data between the Time Points.

Note that any larger DNA fragments (>1 kb) from the starting sample may, in theory, be retained during sample clean-up steps in the library preparation workflow based on size alone and irrespective of the success of adaptor ligation/amplification. Thus, such material may contribute to the fluorescence signal in the library fluorescent quantification assay, contributing to the increased library concentration for later Time Points and extended sample storage time in the study (FIG. 6).

The library concentration data for the 276 data points noted above was processed through a main effects analysis using the variability gauge data quality assessment in JMP to estimate what proportion of the variability in the library concentration data was attributable to the study variable (EDTA storage time) and the runs performed. The variance components results from this test are shown below in Table 6.

### Table 6 Variance Components Analysis of the Library Concentration Data by Run Number and Time Point for the 276 Samples Analysed in the Study

Variance components analysis showed that the study variable—EDTA storage time (Time Point)—contributed 50% of the variance seen in the library concentration data (Table 6). Thus, 50% of the variance observed in the library concentration data is from other workflow variables; 10% of this variance is attributed to run-to-run variability, with the remaining 40% attributed to variables not specifically investigated in this analysis/study (Table 6 Table 6). Possible variables may include, but are not limited to, multiple operators performing sample centrifugation and plasma isolation at the varying Time Points, lab/workflow variability across Runs 1-9 (e.g., Service Lab Runs 1-3 vs R&D Runs 4-9) as well as variability relating to the patients themselves from whom the samples were collected.

### Sequencing Validity

To assess the quality of DNA sample and omit any samples that had an issue with the sequencing process that effected the quality of the sequence results, each sequenced sample was analysed for several parameters, primarily, run control and sequencing quality control of the whole run, fragment count, fragment size profile (as compared to what is expected for cfDNA size selected sequencing library), GC profile fetal fraction, and consistency checks. All samples must have at least ≥2% fetal fraction. In addition, all samples at risk of a false negative or false positive result are evaluated using our proprietary dynamic fetal fraction assessment. This adapts the level of required fetal fraction for the sample to the quality and quantity of the supporting sequencing data.

The overall sequencing validity rate for all samples tested (11 samples had one retest) was 272/274 (99.27%). Eleven (11) samples which initially failed sample validity were retested on a subsequent run; nine (9) of these samples returned a valid result on retesting.

The initial and 1× retest sequencing validity rates across each Time Point are presented below in Table 7Error! Reference source not found.

TABLE 7
Initial sequencing validity and 1x retest rates for across each Time Point and the overall
validity rate as a part of this study.

| | Time Point | Time Point | Time Point | Time Point | |
|---|---|---|---|---|---|
| | 1 | 2 | 3 | 4 | Overall |
| Sequencing Validity | 64/64 | 66/66 | 69/72 | 60/74 | 259/276 |
| Sequencing Validity | 100% | 100% | 95.83% | 81.08% | 93.84% |
| Rate | | | | | |
| Sequencing Validity | 64/64 | 66/66 | 69/72 | 69/74 | 268/276 |
| after 1x retest | | | | | |
| Sequencing Validity | 100% | 100% | 95.83% | 93.24% | 97.10% |
| Rate after 1x retest | | | | | |

Time Point 1 = 0-8 hours, Time Point 2 = 72 hours, Time Point 3 = 120 hours and Time Point 4 = 168 hours

After 1× retest, a total of eight (8) samples failed sequencing validity checks.

Of the six (6) samples that failed sequencing that were not retested:
2× sequencing failures were due to fragment size profile failure at the 160 bp position (M080, Time Point 4 and M085. Time Point 4)
4× sequencing failures were due to fragment size profile failure at the 180 bp position (M089, Time Point 4; M101, Time Point 3; M101, Time Point 4 and M102, Time Point 4)
   Of the two (2) samples that failed sequencing validity after 1× retest:
1× sequencing failure was due to fragment size profile failure at the 180 bp position (M068, Time Point 3)
1× sequencing failure was due to fragment size profile failure at both the 120 bp and 180 bp position (M076, Time Point 3)

An increased number of samples failing the sequencing QC checks at later Time Points (particularly the ‘Abnormal Fragment Size Profile’ check) is likely due to the increased DNA input as a result of extended sample storage time in the EDTA tubes resulting in PCR plateau and heteroduplex formation of library fragments in latter PCR cycles. Thus, the sequencing of heteroduplex library fragments after sample enrichment by size selection (on the QS250) is expected to increase the risk of a sample failing for the IONA software QC checks; indeed, Time Point 4 has the highest occurrence samples failing initial sequencing validity and after 1× retest (Table), most commonly for the ‘Abnormal Fragment Size Profile’. Nevertheless, repeating the sample through library preparation and sequencing was able generate a valid result.

An example of the shift in fragment size frequency concomitant with prolonged storage time prior to centrifugation can be observed in
for two patients that returned a valid result for each Time Point.

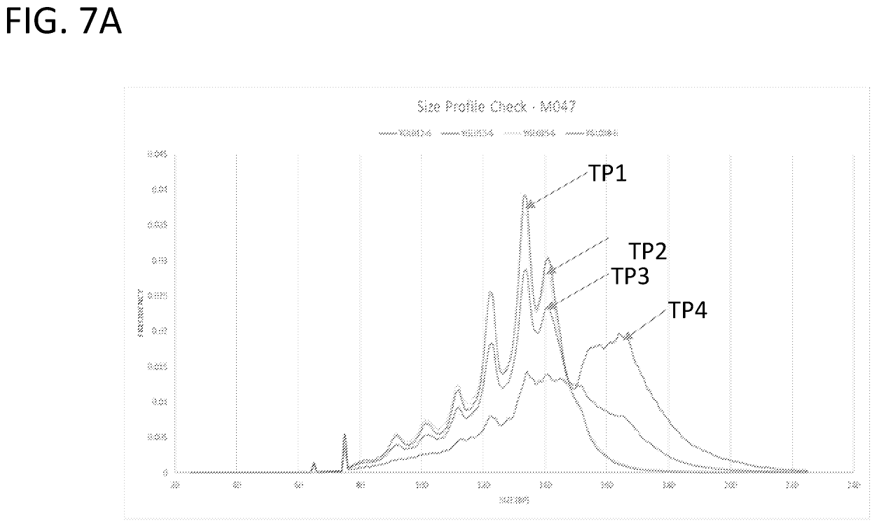

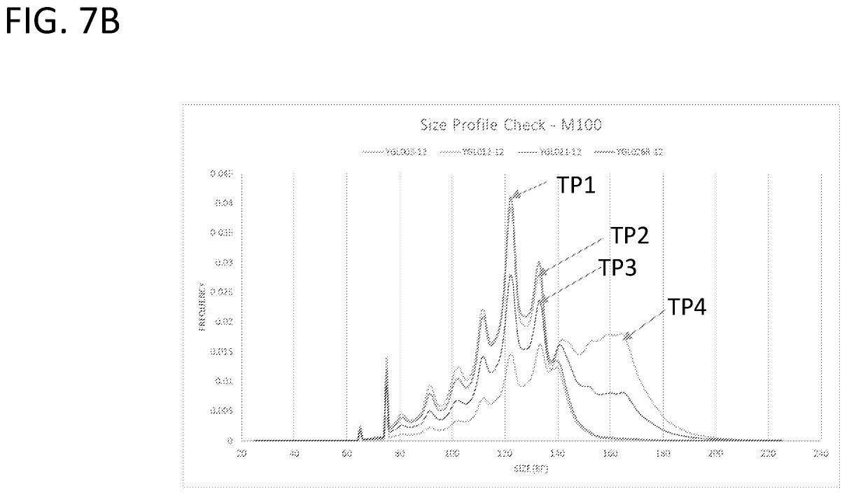

The representative shift in fragment profiles to the right (i.e., larger DNA library insert sizes) demonstrated in FIGS. 7A and 7B for patient samples M047 and M100 over all four Time Points is the expected result of heteroduplex formation; itself a consequence of increased DNA input into library preparation from the extended sample storage time in EDTA tubes. It should be noted that all aligned DNA fragments represented by the profiles in FIGS. 7A and 7B are within the expected size of the mono-nucleosome cfDNA units and not expected DNA fragments resulting from cell lysis as a result of extended storage times prior to centrifugation and plasma isolation.

### Fetal Fraction Percentage

The “IONA FF” method applies one of two algorithms to produce the estimate, depending on values measured from the chromosome count analysis. IONA FF looks at X and Y chromosome proportions. Then.
If there is a very high degree of confidence that a sample is fetal male, then it uses the ‘E1’ estimator algorithm. This uses a linear model to estimate FF from X and Y chromosome data, with the model having been calibrated during development.
Otherwise (i.e. can't be confident it's fetal male), it uses the ‘E2’ algorithm for a sample. This method estimates from a model that relates relative frequencies of long and short fragments to FF. This is also a linear model, but is calibrated per-run using the already-calculated E1 (X/Y-based) estimates. This increases accuracy of size-based estimates vs. using a static model, by removing run-to-run variation in the measurement process.
   The estimate is then output to the rest of the system, together with an internal estimate of the measurement uncertainty, that is also used by the validity checkers. Another way to calculate fetal fraction is with the algorithm well known in the field as SeqFF.

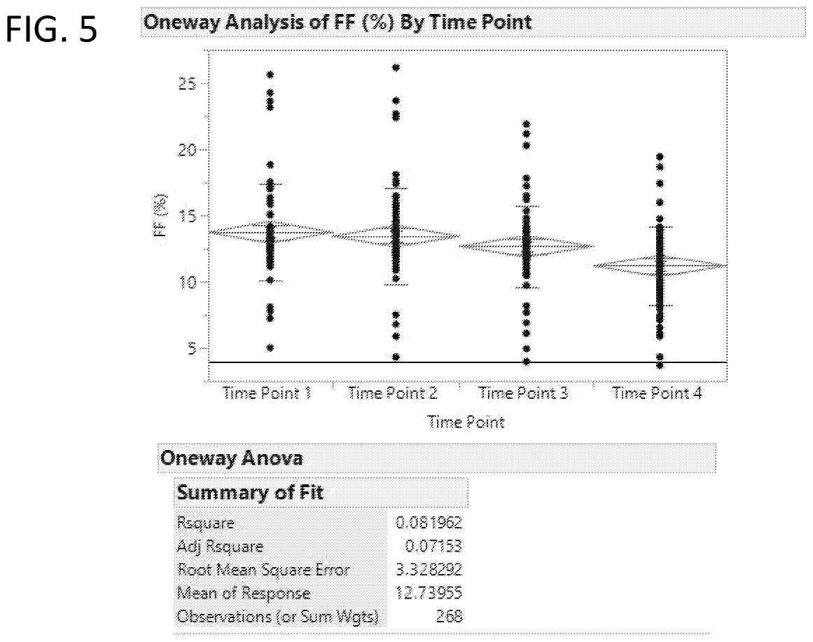

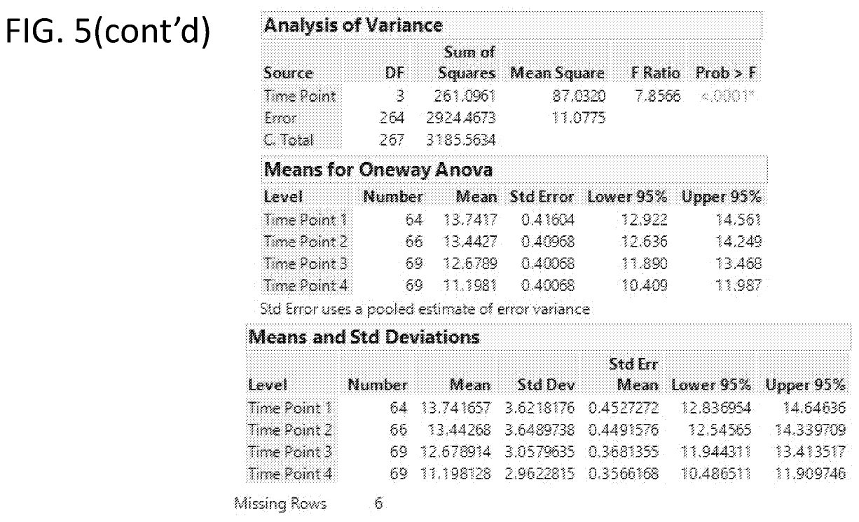

Fetal fraction percentage was determined from the data of all 268 valid samples after analysis by the IONA software. One-way ANOVA was also performed on the raw fetal fraction data (the enriched fetal fraction estimates obtained from the raw sequencing data) to assess the comparison between each Time Point. ANOVA of all fetal fraction data is shown in FIG. 5.

Visually, the data demonstrates a trend of shifted lower fetal fraction percentage concomitant with extended storage time prior to centrifugation. All Time Points presented with similar standard deviations when analysing all 268 fetal fraction estimates, supported by the unequal variance test noted above, suggesting there is comparable variance across these Time Points.

The ANOVA p-value of <0.0001 in FIG. 5 indicates there is a significant variance in the fetal fraction percentage between the Time Points.

Notwithstanding, 267 of the 268 samples have a fetal fraction greater than 4%, a common threshold applied for NIPT tests, and all passed the IONA Nx FF cut off.

### Rare Autosomal Aneuploidy Analysis

All 272 (100%) valid samples returned a valid RAA plugin result of ‘Not Detected’, indicating no aneuploidy was detected.

### Concordance Analysis

Concordance analysis across each Time Point for valid samples within this study was performed only where a valid baseline result (Time Point 1) was returned, even in the absence of a result for another respective Time Point (i.e., resulting from library or sequencing failure), where an invalid result in this context refers to a ‘Not Calculated’ result in reference to SCA/FSD determination. Concordance analysis was also performed for patient samples that returned a valid result for all four Time Points.

### RAA Concordance Analysis

The concordance analysis for RAA status comparative to the baseline (Time Point 1) is summarised below in Table 11. Samples were considered where a valid Time Point 1 RAA result was available alongside at least one other Time Point.

TABLE 11
Concordance analysis for RAA status comparative to
the valid baseline (Time Point 1) result.

| | Time | Time | Time | Time |
|---|---|---|---|---|
| | Point 1 | Point 2 | Point 3 | Point 4 |
| Valid RAA | 64 | 60/64 | 62/64 | 59/64 |
| Result | | | | |
| # Valid for | | 60 | 62 | 59 |
| Concordance | | | | |
| Analysis | | | | |
| Concordant | N/A | 60/60 | 62/62 | 59/59 |
| RAA VS Time | | | | |
| Point 1 | | | | |
| Concordant | N/A | 100% | 100% | 100% |

Time Point 2—60/60 (100%) patient samples returned a ‘Not Detected’ RAA result concordant with the baseline.
Time Point 3—62/62 (100%) patient samples returned a ‘Not Detected’ RAA result concordant with the baseline.
Time Point 4—59/59 (100%) patient samples returned a ‘Not Detected’ RAA result concordant with the baseline.

The concordance analysis for RAA status comparative to the baseline of fifty-five (55) patient samples that returned a valid result for all four Time Points is summarised below in Table 12.

TABLE 12
Concordance analysis for RAA status comparative to the
valid baseline result for fifty-five (55) patient
samples that returned a valid result for all Time Points.

| | Time | Time | Time | Time |
|---|---|---|---|---|
| | Point 1 | Point 2 | Point 3 | Point 4 |
| Valid RAA | 55 | 55/55 | 55/55 | 55/55 |
| Result | | | | |
| Concordance | N/A | 100% | 100% | 100% |

Time Point 2—55/55 (100%) samples returned a ‘Not Detected’ RAA result concordant with the baseline.
Time Point 3—55/55 (100%) samples returned a ‘Not Detected’ RAA result concordant with the baseline.
Time Point 4—55/55 (100%) samples returned a ‘Not Detected’ RAA result concordant with the baseline.

### T13/18/21 Concordance Analysis

The concordance analysis for T13/18/21 aneuploidy status comparative to the baseline (Time Point 1) is summarised below in

Table.

TABLE 13
Concordance analysis for T13/18/21 status comparative to
the valid baseline (Time Point 1) result.

| | Time | Time | Time | Time |
|---|---|---|---|---|
| | Point 1 | Point 2 | Point 3 | Point 4 |
| Valid | 64 | 60/60 | 62/62 | 59/59 |
| T13/18/21 | | | | |
| Result | | | | |
| # Valid for | | 60 | 62 | 59 |
| Concordance | | | | |
| Analysis | | | | |
| Concordant | N/A | 60/60 | 62/62 | 59/59 |
| T13/18/21 VS | | | | |
| Time Point 1 | | | | |
| Concordance | N/A | 100% | 100% | 100% |

Time Point 2—60/60 (100%) patient samples returned a ‘Low Risk’ result concordant with the baseline.
Time Point 3—62/62 (100%) patient samples returned a ‘Low Risk’ result concordant with the baseline.
Time Point 4—59/59 (100%) patient samples returned a ‘Low Risk’ result concordant with the baseline.

The concordance analysis for T13/18/21 aneuploidy status comparative to the baseline of fifty-five (55) patient samples that returned a valid result for all four Time Points is summarised below in Table.

TABLE 14
Concordance analysis for T13/18/21 status comparative to the
valid baseline result for fifty-five (55) patient samples
that returned a valid result for all Time Points.

| | Time | Time | Time | Time |
|---|---|---|---|---|
| | Point 1 | Point 2 | Point 3 | Point 4 |
| Valid | 55/55 | 55/55 | 55/55 | 55/55 |
| T13/18/21 | | | | |
| Result | | | | |
| Valid for | N/A | 55 | 55 | 55 |
| Concordance | | | | |
| Analysis | | | | |
| Concordance | N/A | 100% | 100% | 100% |

Time Point 2—55/55 (100%) samples returned a ‘Low Risk’ result concordant with the baseline.
Time Point 3—55/55 (100%) samples returned a ‘Low Risk’ result concordant with the baseline.
Time Point 4—55/55 (100%) samples returned a ‘Low Risk’ result concordant with the baseline.

### Example 4

This example provides an embodiment of a parameter of a sequence validity check that looks at the fragment size profile and makes a determination if the profile is normal or abnormal. The purpose of this check is to reduce the risk of performance degradation in an NIPT test due to sample degredation and/or reduced effectiveness of a fetal DNA size selection step (also referred to as fetal DNA enrichment), by detecting cases of insufficient enrichment and invalidating corresponding sample analyses such that they do not produce a test result. The test can also be used to test for cancer cell sequences in the cell free DNA.

Fetal cell-free DNA is known to be present in a specific size range which is different from that of maternal cell-free DNA. Therefore, it is possible to detect cases of ineffective enrichment and/or excessive sample degredation by computing a normalised fragment size profile from sequencing data associated with a sample, and comparing its parameters against a pre-calculated reference parameter set which can distinguish effective from ineffective enrichment and/or excessive sample degradation.

The Size Profile Validity Check is designed to ensure, for example, that:
1. Enrichment of the fetal cell-free DNA component of maternal cell-free DNA is effective; enrichment may be applied through size selection as described herein, by ensuring that fragments in size ranges more likely to be fetal are preferentially selected and/or
2. Sample sequencing data possesses a DNA fragment size profile consistent with the biological characteristics of cell-free DNA material extracted from plasma drawn from a pregnant woman.

One or more size envelopes are defined at particular fragment size values of interest. (In Example 3, the size fragment size values of interest were 120 bp and 180 bp fragments.) Essentially, each envelope encloses a fragment size profile at those points which is considered to be valid. Therefore, if an incoming fragment size profile meets the conditions for enclosure by at least one of the envelopes so defined, the Size Profile Validity Check is considered to have passed for the corresponding sample.

Configuration Parameters: The following parameters are required in order to configure this embodiment of the validity check parameter. They should be stored in an efficient hierarchical structured representation (for example, using an XML-based format).
Number of size envelopes defined—Nₑₙᵥ (integer, mandatory). 108,
Array of size envelopes—SizeEnv[⋅]. For each entry:
   Number of size checkpoints—N_{chk} (integer, mandatory).
   Array of size checkpoints—SizeChk[⋅]. For each entry:
      Fragment size for which defined—s_{chk} (integer, mandatory).
      Valid relative frequency range—lower limit—z_{L} (real, optional). Note: Provided that a value is supplied for z_{U}, this value may be omitted, in which case the lower limit is to be taken as being zero.
      Valid relative frequency range—upper limit—z_{U} (real, optional). Note: Provided that a value is supplied for z_{L}, this value may be omitted, in which case the upper limit is to be taken as being one.

Test Input Data: The input data is an array of fragment size values measured in sequencing data.

Test Output: The method produces a single output, as follows: Size Profile Validity Status—V_{SP} (Boolean, mandatory).

Calculation of Sample Size Profile: First, a relative frequency distribution is formed from the array of fragment size measurement values produced for the sample, as generated by the analysis core pipeline via a method appropriate to the sequencing platform in use. This distribution is to be computed as an array composed of the frequency of each size which occurs, normalised according to the total number of fragments included in the distribution, thus:

$$Z [ s ] = \frac{{n}_{s}}{{\sum }_{j ∈ S} {n}_{j}}$$

where nₛ is the number of fragments present of size s for all fragment sizes s∈S, and S is the set of all fragment sizes measured from the sequencing data, where contributing fragments are considered to be only those that (following de-duplication) have aligned uniquely against a chromosome other than 13, 18, 21, X and Y. Note that where N fragments were found to align at the same genomic location, and thus de-duplication tool place, each fragment of size s will contribute 1/N to the fragment count nₛ.

For any size value not represented (i.e. s∉S), the corresponding relative frequency distribution value Z[s] is taken to be zero.

Calculation of sample validity status: The relative frequency distribution Z computed for the sample is compared against each of the envelopes defined in the configuration parameter set in turn. For each envelope, this comparison proceeds as follows:
1. At each size checkpoint, the corresponding size value s_{chk} is used to look up a value for relative frequency at that size in the computed distribution for the sample, viz: Z[s_{chk}].
2. The distribution value so obtained is compared against the valid frequency range parameters, z_{L} and z_{U}. If it does not fall within the interval [z_{L}, z_{U}), that is: Z[s_{chk}]<z_{L} or Z[s_{chk}]≥z_{U}, then the comparison for the checkpoint is marked as having failed. If the distribution value does fall within the interval [z_{L}, z_{U}), the comparison is marked as having passed.

The comparison proceeds to the next size checkpoint in the present envelope until none remain.

A comparison result for the envelope is then calculated as the logical ‘and’ of all constituent checkpoint results. That is, an envelope comparison is considered to have passed only if it has passed for all constituent checkpoints.

If any single envelope comparison is found to have passed, a valid result is generated for sample validity via the Size Profile Validity Check, and it terminates with a value V_{SP}=true. Only one envelope comparison is required to succeed in order for the validity check as a whole to give a valid result.

If, after all envelope comparisons have completed it is found that none have passed for all constituent checkpoints, an invalid result is generated for sample validity via the Size Profile Validity Check, and it terminates with a value V_{SP}=false. This equates to an abnormal fragment size profile and the sample does not pass the sequence validity check and is rejected. A blood redraw is required.

### Equivalents

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications mentioned in this specification are herein incorporated by reference into the specification to the same extent as if each individual publication was specifically and individually indicated to be incorporated herein by reference in their entireties.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method for analyzing nucleic acid sequences obtained from a blood sample to provide sequence information, said method comprising the step of:
a. analyzing nucleic acid sequences of size-selected cell free DNA to detect sequence information,
wherein the size-selected cell free DNA was obtained from a blood sample,
wherein the blood sample was collected in a blood collection device that did not comprise a fixative,
wherein the blood sample was stored without a fixative at a temperature greater than −20° C. and less than 35° C. prior to the cell free DNA being isolated from the plasma,
wherein the plasma was separated from the blood cells present in the blood sample either before or after storage,
wherein cell free DNA was isolated from the plasma at least 48 hours after the blood sample was collected from a subject, and
wherein the size-selected cell free DNA being analyzed was obtained by separating by size the extracted cell free DNA and isolating the cell free DNA of the extracted cell free DNA that is less than 300 bp.

2. A method for analyzing nucleic acid sequences obtained from a blood sample to provide sequence information, said method comprising the step of:
a. analyzing nucleic acid sequences of size-selected cell free DNA to detect sequence information,
wherein the size-selected cell free DNA was obtained from a blood sample,
wherein the blood sample was collected in a blood collection device comprising a container having only one or more compositions therein wherein the one or more compositions consists of one or more anti-coagulants and a metal ion selected from potassium, lithium, and sodium,
wherein the blood sample was stored in the blood collection device at a temperature greater than −20° C. and less than 35° C. prior to the cell free DNA being isolated from the plasma,
wherein the plasma was separated from the blood cells present in the blood sample either before or after storage,
wherein cell free DNA was isolated from the plasma at least 48 hours after the blood sample was collected from a subject, and
wherein the size-selected cell free DNA being analyzed was obtained by separating by size the extracted cell free DNA and isolating the cell free DNA of the extracted cell free DNA that is less than 300 bp.

3. A method for analyzing nucleic acid sequences obtained from a blood sample to provide sequence information, said method comprising the step of:
a. analyzing nucleic acid sequences of size-selected cell free DNA to detect sequence information,
wherein the size-selected cell free DNA was obtained from a blood sample,
wherein the blood sample was collected in a blood collection device comprising a container having only one or more compositions therein wherein the one or more compositions consists of one or more anti-coagulants and a metal ion selected from potassium, lithium, and sodium; and a separator gel,
wherein the blood sample was stored in the blood collection device at a temperature greater than −20° C. and less than 35° C. prior to the cell free DNA being isolated from the plasma,
wherein the plasma was separated from the blood cells present in the blood sample either before or after storage,
wherein cell free DNA was isolated from the plasma at least 48 hours after the blood sample was collected from a subject, and
wherein the size-selected cell free DNA being analyzed was obtained by separating by size the extracted cell free DNA and isolating the cell free DNA of the extracted cell free DNA that is less than 300 bp.

4. The method of claim 1, wherein the cfDNA was isolated from the plasma at least 3, 4, 5, 6, 7, or 8 days after blood collection.

5. The method of claim 1, wherein the plasma was separated from the blood after storage of at least 2, 3, 4, 5, 6, 7, or 8 days after blood collection.

6. (canceled)

7. The method of claim 4, wherein the blood sample was stored in the blood collection device at a temperature greater than 0° C. and less than 35° C. or less than 30° C. or less than 25° C. or less than 20° C. prior to the cell free DNA being isolated from the plasma.

8. The method of claim 5, wherein the blood sample was stored in the blood collection device at a temperature greater than 0° C. and less than 35° C. or less than 30° C. or less than 25° C. or less than 20° C. prior to the plasma being separated from the blood.

9. The method of claim 4, wherein the blood sample was stored in the blood collection device at a temperature greater than 0° C. or greater than 2° C. and less than 15° C. or less than 10° C. or less than 8° C. or less than 6° C. prior to the cell free DNA being isolated from the plasma.

10. The method of claim 5, wherein the blood sample was stored in the blood collection device at a temperature greater than 0° C. or greater than 2° C. and less than 15° C. or less than 10° C. or less than 8° C. or less than 6° C. prior to the plasma being separated from the blood.

11. The method of claim 5, wherein the blood sample was stored in the blood collection device at ambient temperature prior to the plasma being isolated from the blood.

12. The method of claim 1, wherein nucleic acid sequences of the isolated cell free DNA is analyzed to determine the presence of a genetic anomaly in fetal DNA.

13. The method of claim 1, wherein the sequence information comprises the sequence of a plurality of sequence tags and the method further comprises
a. analyzing the sequences of the plurality of sequence tags to determine the presence of at least one sequence of interest in the fetus's DNA, wherein at least a portion of the plurality of sequence tags map to the at least one sequence of interest.

14. The method of claim 1, wherein sequence information comprises a genetic mutation in a gene;
optionally, wherein said gene is selected from the group consisting of: BRCA1, BRCA2, MSH6, MSH2, MLH1, RET, PTEN, ATM, H-RAS, p53, ELAC2, CDH1, APC, AR, PMS2, MLH3, CYP1A1, GSTP1, GSTM1, AXIN2, CYP19, MET, NAT1, CDKN2A, NQ01, trc8, RAD51, PMS1, TGFBR2, VHL, MC4R, POMC, NROB2, UCP2, PCSK1, PPARG, ADRB2, UCP3, glur1, cart, SORBS1, LEP, LEPR, SIM1, TNF, IL-6, IL-1, IL-2, IL-3, IL1A, TAP2, THPO, THRB, NBS1, RBM15, LIE, MPL, RUNX1, Her-2, glucocorticoid receptor, estrogen receptor, thyroid receptor, p21, p27, K-RAS, N-RAS, retinoblastoma protein, Wiskott-Aldrich (WAS) gene, Factor V Leiden, Factor II (prothrombin), methylene tetrahydrofolate reductase, cystic fibrosis, LDL receptor, HDL receptor, superoxide dismutase gene, and SHOX gene or said gene is selected from genes involved in nitric oxide regulation, genes involved in cell cycle regulation, tumor suppressor genes, oncogenes, and genes associated with neurodegeneration U; or
optionally, wherein the genetic mutation is a marker for a type of cancer.

15. (canceled)

16. (canceled)

17. (canceled)

18. The method of claim 1, wherein the portion of the cfDNA from the plasma sample is less than 155 bp or less than 150 bp.

19. The method of claim 18, wherein the portion of the cfDNA from the plasma sample is greater than 50 bp or greater than 80 bp or greater than 90 bp or greater than 100 bp.

20. The method of claim 1, wherein the fixative is a crosslinking agent, metabolic inhibitor, or a membrane stabilizer.

21. The method of claim 20, wherein the collection device comprises EDTA and a metal ion selected from potassium.

22. The method of claim 21, wherein the collection device further comprises a separator gel.

23. (canceled)

24. (canceled)

25. The method of claim 1, wherein a sequence library was generated comprising the cell free DNA before or after the cfDNA was isolated and separated by size.

26. (canceled)

27. The method of claim 1, wherein the separation step was performed using a bead-based binding matrix, column chromatography, or a membrane filter or wherein the separation step was performed using gel electrophoresis.

28. (canceled)

29. (canceled)

30. (canceled)

31. (canceled)

32. The method of claim 1, wherein analyzing nucleic acid sequences of the isolated cell free DNA to detect sequence information comprises sequencing the isolated cell free DNA or sequencing at least a portion of the cell free DNA to determine the nucleic acid sequence of one or more polynucleotides in the isolated cell free DNA.

33. The method of claim 1, wherein the sequence information comprises a nucleic acid sequence having an insertion, deletion, chromathripsis, or methylation as compared to a reference sequence or wherein nucleic acid sequences of the isolated cell free DNA are analyzed for an abnormal number of chromosomes or for the presence of an XX or an XY chromosome by calculating chromosome ratios.

34. (canceled)

35. The method of claim 1 further comprising a step of creating a first normalised fragment size profile from the sequencing information associated with the sample, comparing one or more values within the fragment size profile against one or more of corresponding value ranges obtained from a reference parameter set comprising a plurality of valid fragment size profiles: and accepting the sequence information and the analysis results thereof if one of the one or more values within the fragment size profile associated with the sample is within the corresponding value range or rejecting the sequence information if one of the one or more values within the fragment size profile associated sample is outside of the corresponding value range.

36. The method of claim 35, wherein the first normalized fragment size profile comprises or consists of fragment sizes less than 185 bp and greater than 50 bp.

37. (canceled)

38. The method of claim 3 further comprising for the sample with rejected sequence information, a step of submitting a request for a redraw of a sample from the subject from which the sample with rejected sequence information was obtained.

39. The method of claim 1 further comprising a step of calculating a first relative fragment size frequency value from the sequencing information associated with the sample; comparing the first relative fragment size frequency value to a reference relative fragment size frequency set comprising a plurality of valid relative fragment size frequencies, and accepting the sequence information and the analysis results thereof if the first fragment size frequency associated with the sample is within a specified range of values within the reference set or rejecting the sequence information and the analysis results thereof if the first fragment size frequency associated with the sample is outside a specified range of values within the reference set.

40. The method of claim 39, wherein the first relative fragment size frequency value is a fragment size less than 185 bp and greater than 50 bp.

41. (canceled)

42. The method of claim 39 further comprising, for the sample with rejected sequence information, a step of submitting a request for a redraw of a sample from the subject from which the sample with rejected sequence information was obtained.

43. The method of claim 39 further comprising a step of performing the calculating and comparing steps of claim 39 for a second relative fragment size frequency value of a different bp value than the first fragment size frequency, wherein the sequence information and the analysis thereof is accepted if the second fragment size frequency value associated with the sample is within a specified range of values within the reference set or rejecting the sequence information if all the second fragment size frequency associated with the sample is outside a specified range of values within the reference set.
